(19) United States
(12) Patent Application Publication
(10) Pub. No.: US 2025/0243529 A1
(43) Pub. Date: Jul. 31, 2025

(54) METHODS FOR MULTIPLEX DETECTION OF SENSE AND ANTISENSE STRANDS IN AN OLIGONUCLEOTIDE DUPLEX

(71) Applicant: MESO SCALE TECHNOLOGIES, LLC., Rockville, MD (US)

(72) Inventors: Rohini DATTA, Greenbelt, MD (US); Timothy J. BREAK, Clarksburg, MD (US); Seth B. HARKINS, Bethesda, MD (US)

(21) Appl. No.: 19/035,358

(22) Filed: Jan. 23, 2025

Related U.S. Application Data
(60) Provisional application No. 63/624,974, filed on Jan. 25, 2024.

(51) Int. Cl. C12Q 1/6834; C12Q 1/44; C12Q 1/6818

(52) U.S. Cl. CPC C12Q 1/6834; C12Q 1/44; C12Q 1/6818; G01N 2333/922

(57) ABSTRACT

Described herein is a method for detecting an oligonucleotide in a sample, and in particular, to a method for detecting sense and antisense strands of an oligonucleotide duplex in a sample.

### FIELD OF THE INVENTION

The present disclosure relates to a method for detecting or quantifying an oligonucleotide in a sample, and in particular, to a method for detecting or quantifying a sense and an antisense strand of an oligonucleotide duplex.

### BACKGROUND OF THE INVENTION

Nucleic acid-based therapeutics make up a class of promising candidates for drug therapy, particularly for biological targets that conventional therapeutics such as small molecule, protein- or antibody-based therapeutics cannot access. Nucleic acid-based therapeutics include single stranded or double stranded oligonucleotide molecules that inhibit DNA or RNA expression, for example, to reduce or prevent production of an abnormal protein associated with a disease. Several nucleic acid-based therapeutics have been approved by the U.S. Food and Drug Administration and more are being investigated in clinical trials for the treatment of a variety of diseases.

Nucleic acids are large molecules that are highly charged, rapidly degraded and cleared from the body, which can result in poor pharmacological properties. Stoddard et al. (2018) “Editorial: Nucleic Acids Research and Nucleic Acid Therapeutics.” Nuc. Acids Res. 46(4):1563-1564. Consequently, pharmacokinetics, tissue targeting, and tissue accumulation are all important considerations when developing nucleic-acid base therapeutics. Highly sensitive and quantitative assay are needed to characterize nucleic acid pharmacokinetics. Thayer et al. (2020) “POE Immunoassay: Plate-based oligonucleotide electrochemiluminescent immunoassay for the quantification of nucleic acids in biological matrices.” Scientific Reports. 10(1):10425 (doi.org/10.1038/s41598-020-66829-6).

Although various polymerase-chain reaction (PCR) based, size-exclusion chromatography (SEC), and liquid chromatography-mass spectrometry (LC-MS) methods exist for characterizing nucleic acid therapeutics, these methods are limited by assay sensitivity and time-consuming extraction steps. Id. The unique biophysical properties of oligonucleotide therapeutics can lead to atypical absorption, distribution, metabolism and elimination (ADME) processes, as well as pharmacokinetics-pharmacodynamics (PKPD) uncoupling. The difficulty in understanding these relationships can lead to inefficiencies throughout the drug development process, including increased animal usage, and may lead to an increased risk in human trials. Id. Quantification of both strands of an oligonucleotide therapeutic can be important to understanding the stability and metabolic pathways of the oligonucleotide therapeutic and to developing models to elucidate the pharmacology of the therapeutic. Id.

As such, there remains a need for sensitive assays for the detection or quantitation of oligonucleotide therapeutics, for example, from a sample obtained from a patient.

### SUMMARY OF THE INVENTION

Described herein is a method of detecting or quantifying a sense and an antisense strand of an oligonucleotide duplex in a sample. In one aspect, the method includes:
(a) contacting the sample with a composition that includes a set of probes, wherein the set of probes includes:
(i) a sense probe that includes a first single stranded oligonucleotide tag that is complementary to at least a portion of a first capture oligonucleotide immobilized on a support surface, a sense binding portion capable of hybridizing to a nucleotide sequence of the sense strand of the oligonucleotide duplex, and a first label; and
(ii) an antisense probe that includes a second single stranded oligonucleotide tag that is complementary to at least a portion of a second capture oligonucleotide immobilized on the support surface, an antisense binding portion capable of hybridizing to a nucleotide sequence of the antisense strand of the oligonucleotide duplex, and a second label,
wherein the sense binding portion of the sense probe has a sense binding length that is shorter than a sense strand length of the sense strand, and
wherein the antisense binding portion of the antisense strand has an antisense binding length that is shorter than an antisense strand length of the antisense strand; and
(b) incubating the probes with the sample to form a hybridization mixture that contains hybridization complexes comprising:
(i) a sense complex comprising the sense probe hybridized with the sense strand of the oligonucleotide duplex; and
(ii) an antisense complex comprising the antisense probe hybridized with the antisense strand of the oligonucleotide duplex;
(c) contacting the support surface with the hybridization mixture under conditions in which the first and second oligonucleotide tags of the hybridization complexes hybridize to the first and second capture oligonucleotides immobilized on the support surface and contacting the hybridization mixture with a single-strand specific nuclease; and
(d) detecting or quantifying the sense and antisense strands of the oligonucleotide duplex based on the presence label immobilized on the support surface.

In one aspect, (c) includes:
(i) contacting the support surface with the hybridization mixture under conditions in which the first and second oligonucleotide tags of the hybridization complexes hybridize to the first and second capture oligonucleotides on the support surface to immobilize the hybridization complexes on the support surface; and
(ii) contacting the immobilized hybridization complexes with a single-strand specific nuclease.

In one aspect, (c) includes:
(i) contacting the hybridization mixture with a single-strand specific nuclease to form a reaction mixture; and
(ii) contacting the support surface with the reaction mixture of (i) under conditions in which the first and second oligonucleotide tags of the sense and antisense probes hybridize to the first and second capture oligonucleotides immobilized on the support surface.

In one aspect, the oligonucleotide tag of the antisense probe hybridizes to a capture oligonucleotide immobilized on the support surface. In one aspect, the oligonucleotide tag of the antisense probe that is part of an antisense complex hybridizes to a capture oligonucleotide immobilized on the support surface. In one aspect, the oligonucleotide tag of the antisense probe is not part of an antisense complex. In one aspect, the oligonucleotide tag of the antisense probe is part of a hybridization complex. In one aspect, the hybridization complex does not include an antisense strand of the oligonucleotide duplex. In one aspect, the hybridization complex is a probe-probe complex. In one aspect, the probe-probe complex includes a single stranded overhang.

In one aspect, the oligonucleotide tag of the sense probe hybridizes to a capture oligonucleotide immobilized on the support surface. In one aspect, the oligonucleotide tag of the sense probe that is part of a sense complex hybridizes to a capture oligonucleotide immobilized on the support surface. In one aspect, the oligonucleotide tag of the sense probe is not part of a sense complex. In one aspect, the oligonucleotide tag of the sense probe is part of a hybridization complex. In one aspect, the hybridization complex does not include a sense strand of the oligonucleotide duplex. In one aspect, the hybridization complex is a probe-probe complex. In one aspect, the probe-probe complex includes a single stranded overhang.

In one aspect, the sense and antisense strands of the oligonucleotide duplex each include, individually, from about 8 to about 50 nucleotides. In one aspect, the sense and antisense strands of the oligonucleotide duplex each include, individually, from about 16 to about 30 nucleotides.

In one aspect, the sense strand of the oligonucleotide duplex includes DNA. In one aspect, the sense strand of the oligonucleotide duplex includes RNA. In one aspect, the antisense strand of the oligonucleotide duplex includes DNA. In one aspect, the antisense strand of the oligonucleotide duplex includes RNA.

In one aspect, the oligonucleotide duplex includes a DNA/DNA duplex. In one aspect, the oligonucleotide duplex includes a RNA/RNA duplex. In one aspect, the oligonucleotide duplex includes a DNA/RNA heteroduplex.

In one aspect, the sense strand, antisense strand or both the sense and antisense strands of the oligonucleotide duplex individually include one or more modified nucleic acids. In one aspect, the sense strand, the antisense strand or both the sense and antisense strands of the oligonucleotide duplex individually include a 5′- or 3′-conjugate. In one aspect, the conjugate includes polyethylene glycol (PEG), N-acetylgalactosamine (GalNAc), a cell penetrating peptide (CPP), α-tocopherol, an aptamer, an antibody, cholesterol, squalene, a fatty acid, or a nucleolipid.

In one aspect, the sense and antisense strand of the oligonucleotide duplex includes a nucleic acid sequence of a microorganism. In one aspect, the sense and antisense strand of the oligonucleotide duplex includes a nucleic acid sequence of a microorganism that is a component of the human microbiome. In one aspect, the microorganism is a bacteria, fungi, protozoa or a virus. In one aspect, the microorganism is a bacteria. In one aspect, the sense strand and the antisense strand of the oligonucleotide duplex comprise 16S rRNA or rDNA from bacteria.

In one aspect, the sense binding length of the sense probe is shorter than the sense strand length of the sense strand by at least 1 nucleotide. In one aspect, the sense binding length is about 10 to about 16 nucleotides in length. In one aspect, the sense binding portion of the sense probe has a 5′ end that aligns with a 3′ end of the sense strand of the oligonucleotide duplex.

In one aspect, the antisense binding length of the antisense probe is shorter than the antisense strand length of the antisense strand by at least 1 nucleotide. In one aspect, the antisense binding length is about 10 to about 16 nucleotides in length. In one aspect, the antisense binding portion of the antisense probe has a 5′ end that aligns with a 3′ end of the antisense strand of the oligonucleotide duplex.

In one aspect, the first oligonucleotide tag has a first oligonucleotide tag length and the first capture oligonucleotide has a first capture oligonucleotide length, and the first oligonucleotide tag length is the same as the first capture oligonucleotide length. In one aspect, the first oligonucleotide tag has a first oligonucleotide tag length and the first capture oligonucleotide has a first capture oligonucleotide length, and the first oligonucleotide tag length is the shorter than the first capture oligonucleotide length.

In one aspect, the second oligonucleotide tag has a second oligonucleotide tag length and the second capture oligonucleotide has a second capture oligonucleotide length, and the second oligonucleotide tag length is the same as the second capture oligonucleotide length. In one aspect, the second oligonucleotide tag has a second oligonucleotide tag length and the second capture oligonucleotide has a second capture oligonucleotide length, and the second oligonucleotide tag length is the shorter than the second capture oligonucleotide length.

In one aspect, the sense probe includes DNA. In one aspect, the sense binding portion of the sense probe includes DNA. In one aspect, the first oligonucleotide tag of the sense probe includes DNA. In one aspect, the sense binding portion and the oligonucleotide tag of the sense probe include DNA. In one aspect, the sense binding portion of the sense probe includes DNA and the oligonucleotide tag of the sense probe includes RNA.

In one aspect, the antisense probe includes DNA. In one aspect, the antisense binding portion of the antisense probe includes DNA. In one aspect, the first oligonucleotide tag of the antisense probe includes DNA. In one aspect, the antisense binding portion and the oligonucleotide tag of the antisense probe include DNA. In one aspect, the antisense binding portion of the antisense probe includes DNA and the oligonucleotide tag of the antisense probe includes RNA.

In one aspect, the sense probe includes RNA. In one aspect, the sense binding portion of the sense probe includes RNA. In one aspect, the first oligonucleotide tag of the sense probe includes RNA. In one aspect, the sense binding portion and the oligonucleotide tag of the sense probe include RNA. In one aspect, the sense binding portion of the sense probe includes RNA and the oligonucleotide tag of the sense probe includes DNA.

In one aspect, the antisense probe includes RNA. In one aspect, the antisense binding portion of the antisense probe includes RNA. In one aspect, the first oligonucleotide tag of the antisense probe includes RNA. In one aspect, the antisense binding portion and the oligonucleotide tag of the antisense probe include RNA. In one aspect, the antisense binding portion of the antisense probe includes RNA and the oligonucleotide tag of the antisense probe includes DNA.

In one aspect, the sense probe, the antisense probe or both include one or more modified nucleic acids. In one aspect, one or more modified nucleotides include a locked nucleic acid (LNA). In one aspect, one or more modified nucleotides are selected from phosphodiester (PO); phosphorothioate (PS); 2′O-methyl (2′OMe); 2′O-methoxyethyl (MOE); peptide nucleic acid (PNA); phosphoroamidate morpholino (PMO); locked nucleic acid (LNA); 2′-deoxy-2′-fluoro (2′-F); or a combination thereof.

In one aspect, the single-strand specific nuclease includes a single-strand specific DNase. In one aspect, the single-strand specific DNase is S1 nuclease, P1 nuclease or Mung Bean nuclease. In one aspect, the single-strand specific nuclease includes a single-strand specific RNase. In one aspect, the single-strand specific RNase is RNase A, RNase H, RNase I, RNase III, RNase L, RNase P, RNase PhyM, RNase T1, RNase T2, RNase U2, RNase V, PNPase, RNase PH, RNase R, RNase D, RNase T, RNaseONE, oligoribonuclease, exoribonuclease I, or exoribonuclease II.

In one aspect, (a)-(c) are performed concurrently. In one aspect, (a)-(c) are performed sequentially.

In one aspect, the hybridization conditions in (b) include:
(i) incubating the probes with the sample a first temperature to denature the sense and antisense strands of the oligonucleotide duplex; and
(ii) incubating the probes with the denatured sense and antisense strands of the oligonucleotide duplex at a second temperature to allow the sense and antisense probes to hybridize to the sense and antisense strands.

In one aspect, hybridization further includes incubating the sense and antisense complexes at a hold temperature of about 2° C. to about 8° C.

In one aspect, the hybridization conditions in (b) include:
(i) incubating the probes with the sample a first temperature from about 60° C. to about 95° C. for about 1 minute to about 15 minutes;
(ii) incubating the probes with the sample at a second temperature from about 10° C. to about 65° C. for about 30 seconds to about 5 minutes; and
(iii) incubating the probes with the sample at a hold temperature from about 2° C. to about 8° C.

In one aspect, the hybridization conditions in (b) include:
(i) incubating the probes with the sample a first temperature of about 95° C. for about 2 minutes;
(ii) incubating the probes with the sample at a second temperature of about 65° C. for about 1 min; and
(iii) incubating the probes with the sample at a hold temperature of about 4° C.

In one aspect, the hybridization conditions include a first temperature transition rate between steps (i) and (ii) of about 1° C./s to about 2° C./s. In one aspect, the hybridization conditions include first temperature transition rate between steps (i) and (ii) of about 1.8° C./s. In one aspect, the hybridization conditions include a second temperature transition rate between steps (ii) and (iii) about 0.05° C./s to about 1 C/s. In one aspect, the hybridization conditions include a second temperature transition rate between steps (ii) and (iii) of about 0.1° C./s.

In one aspect, the probes are incubated with the sample in a buffer that includes diluent 54 or N-PLEX hybridization Buffer 1 or 2.

In one aspect, the sample includes a plurality of oligonucleotide duplexes and the composition in (a) includes a plurality of sets of probes, wherein each set of probes hybridizes with a unique sense or antisense strand of a unique oligonucleotide duplex.

In one aspect, (c) includes incubating the support surface with the sense and antisense complexes for about 15 minutes to about 12 hours at a temperature of about 20° C. to about 40° C. In one aspect, (c) includes incubating the support surface with the sense and antisense complexes for about 1 hour to about 2 hours at a temperature of about 20° C. to about 40° C. In one aspect, the support surface is incubated with the sense and antisense complexes while shaking. In one aspect, (c) includes incubating the support surface with the sense and antisense complexes for about 1 hour at a temperature of about 37° C., while shaking at about 705 rpm.

In one aspect, the composition in (a) includes about 20 pM to about 10 nM sense probe. In one aspect, the composition in (a) includes about 20 pM to about 10 nM antisense probe.

In one aspect, the sample includes a biological sample. In one aspect, the sample includes an untreated biological sample. In one aspect, the sample includes a pretreated biological sample. In one aspect, the sample includes a purified sample. In one aspect, the sample is purified by precipitation, centrifugation, or column chromatography. In one aspect, the sample includes an extracted sample. In one aspect, the sample includes a naturally occurring RNase. In one aspect, the method includes combining the sample with an RNase inhibitor before (a). In one aspect, the sample includes cell-free DNA.

In one aspect, the biological sample includes a fluid obtained from an organism. In one aspect, the biological sample includes whole blood, plasma, serum, urine, feces, breast milk, saliva, or amniotic fluid. In one aspect, the sample includes an environmental sample. In one aspect, the sample includes a manufacturing process sample.

In one aspect, the method has a limit of detection of less than about 200 μg/mL.

In one aspect, the support surface includes one or more electrodes. In one aspect, one or more electrodes include a carbon electrode. In one aspect, one or more electrodes include carbon ink electrodes. In one aspect, one or more electrodes are included in a multi-well plate. In one aspect, each well of the multi-well plate includes an electrode.

In one aspect, the label includes a member of a binding pair. In one aspect, the label includes biotin.

In one aspect, the label includes an electrochemiluminescent (ECL) label. In one aspect, the method includes a step of generating an assay signal by contacting the electrodes with an electrochemiluminescence read buffer that includes an electrochemiluminescence co-reactant, and applying an electrical potential to the electrodes. In one aspect, the co-reactant is selected from a tertiary amine, tripropylamine, N-butyldiethanolamine, and combinations thereof.

In one aspect, a composition is provided that includes a set of probes. In one aspect, the set of probes includes:
(a) a sense probe that includes a first single stranded oligonucleotide tag that is complementary to at least a portion of a first capture oligonucleotide, a sense binding portion capable of hybridizing to a nucleotide sequence of a sense strand of an oligonucleotide duplex, and a first label; and
(b) an antisense probe that includes a second single stranded oligonucleotide tag that is complementary to at least a portion of a second capture oligonucleotide, an antisense binding portion capable of hybridizing to a nucleotide sequence of an antisense strand of the oligonucleotide duplex, and a second label,
   wherein the sense binding portion of the sense probe has a sense binding length that is shorter than a sense strand length of the sense strand, and wherein the antisense binding portion of the antisense strand has an antisense binding length that is shorter than an antisense strand length of the antisense strand.

In one aspect, a composition is provided that includes:
(a) an oligonucleotide duplex that includes a sense strand and an antisense strand; and
(b) a set of probes that includes:
   (i) a sense probe that includes a first single stranded oligonucleotide tag that is complementary to at least a portion of a first capture oligonucleotide, a sense binding portion capable of hybridizing to a nucleotide sequence of a sense strand of an oligonucleotide duplex, and a first label; and
   (ii) an antisense probe that includes a second single stranded oligonucleotide tag that is complementary to at least a portion of a second capture oligonucleotide, an antisense binding portion capable of hybridizing to a nucleotide sequence of an antisense strand of the oligonucleotide duplex, and a second label,
   wherein the sense binding portion of the sense probe has a sense binding length that is shorter than a sense strand length of the sense strand, and wherein the antisense binding portion of the antisense strand has an antisense binding length that is shorter than an antisense strand length of the antisense strand.

In one aspect, a composition is provided that includes one or more hybridization complexes. In one aspect, the hybridization complexes include:
(a) a sense complex that includes a sense probe hybridized to a sense strand of an oligonucleotide duplex, wherein the sense probe includes a first single stranded oligonucleotide tag that is complementary to at least a portion of a first capture oligonucleotide, a sense binding portion capable of hybridizing to a nucleotide sequence of the sense strand of the oligonucleotide duplex, and a first label, wherein the sense binding portion of the sense probe has a sense binding length that is shorter than a sense strand length of the sense strand;
(b) an antisense complex that includes an antisense probe hybridized to an antisense strand of the oligonucleotide duplex, wherein the antisense probe includes a second single stranded oligonucleotide tag that is complementary to at least a portion of a second capture oligonucleotide, an antisense binding portion capable of hybridizing to a nucleotide sequence of the antisense strand of the oligonucleotide duplex, and a second label, wherein the antisense binding portion of the antisense strand has an antisense binding length that is shorter than an antisense strand length of the antisense strand,
and combinations thereof.

In one aspect, the sense and antisense strands of the oligonucleotide duplex in the composition each include, individually, from about 8 to about 50 nucleotides. In one aspect, the sense and antisense strands of the oligonucleotide duplex in the composition each include, individually, from about 16 to about 30 nucleotides.

In one aspect, the sense strand of the oligonucleotide duplex in the composition includes DNA. In one aspect, the sense strand of the oligonucleotide duplex includes RNA. In one aspect, the antisense strand of the oligonucleotide duplex in the composition includes DNA. In one aspect, the antisense strand of the oligonucleotide duplex includes RNA. In one aspect, the oligonucleotide duplex includes a DNA/DNA duplex. In one aspect, the oligonucleotide duplex includes a RNA/RNA duplex. In one aspect, the oligonucleotide duplex includes a DNA/RNA heteroduplex.

In one aspect, the sense strand, antisense strand or both the sense and antisense strands of the oligonucleotide duplex in the composition individually include one or more modified nucleic acids. In one aspect, the sense strand, the antisense strand or both the sense and antisense strands of the oligonucleotide duplex in the composition individually include a 5′- or 3′-conjugate. In one aspect, the conjugate includes polyethylene glycol (PEG), N-acetylgalactosamine (GalNAc), a cell penetrating peptide (CPP), α-tocopherol, an aptamer, an antibody, cholesterol, squalene, a fatty acid, or a nucleolipid.

In one aspect, the sense binding length of the sense probe in the composition is shorter than the sense strand length of the sense strand by at least 1 nucleotide. In one aspect, the sense binding length of the sense probe in the composition is about 10 to about 16 nucleotides in length. In one aspect, the sense binding portion of the sense probe in the composition has a 5′ end that aligns with a 3′ end of the sense strand of the oligonucleotide duplex.

In one aspect, the antisense binding length of the antisense probe in the composition is shorter than the antisense strand length of the antisense strand by at least 1 nucleotide. In one aspect, the antisense binding length of the antisense probe in the composition is about 10 to about 16 nucleotides in length. In one aspect, the antisense binding portion of the antisense probe in the composition has a 5′ end that aligns with a 3′ end of the antisense strand of the oligonucleotide duplex.

In one aspect, the sense probe in the composition includes DNA. In one aspect, the sense binding portion of the sense probe includes DNA. In one aspect, the first oligonucleotide tag of the sense probe includes DNA. In one aspect, the sense binding portion and the oligonucleotide tag of the sense probe include DNA. In one aspect, the sense binding portion of the sense probe includes DNA and the oligonucleotide tag of the sense probe includes RNA.

In one aspect, the antisense probe in the composition includes DNA. In one aspect, the antisense binding portion of the antisense probe includes DNA. In one aspect, the first oligonucleotide tag of the antisense probe includes DNA. In one aspect, the antisense binding portion and the oligonucleotide tag of the antisense probe include DNA. In one aspect, the antisense binding portion of the antisense probe includes DNA and the oligonucleotide tag of the antisense probe includes RNA.

In one aspect, the sense probe in the composition includes RNA. In one aspect, the sense binding portion of the sense probe includes RNA. In one aspect, the first oligonucleotide tag of the sense probe includes RNA. In one aspect, the sense binding portion and the oligonucleotide tag of the sense probe include RNA. In one aspect, the sense binding portion of the sense probe includes RNA and the oligonucleotide tag of the sense probe includes DNA.

In one aspect, the antisense probe in the composition includes RNA. In one aspect, the antisense binding portion of the antisense probe includes RNA. In one aspect, the first oligonucleotide tag of the antisense probe includes RNA. In one aspect, the antisense binding portion and the oligonucleotide tag of the antisense probe include RNA. In one aspect, the antisense binding portion of the antisense probe includes RNA and the oligonucleotide tag of the antisense probe includes DNA.

In one aspect, the sense probe, the antisense probe or both probes in the composition include one or more modified nucleic acids. In one aspect, one or more modified nucleotides include a locked nucleic acid (LNA). In one aspect, one or more modified nucleotides are selected from phosphodiester (PO); phosphorothioate (PS); 2′O-methyl (2′OMe); 2′O-methoxyethyl (MOE); peptide nucleic acid (PNA); phosphoroamidate morpholino (PMO); locked nucleic acid (LNA); 2′-deoxy-2′-fluoro (2′-F) or a combination thereof.

In one aspect, the label in the composition includes a member of a binding pair. In one aspect, the label includes biotin. In one aspect, the label in the composition includes an electrochemiluminescent label.

In one aspect, a kit is provided for carrying out the method described herein.

### BRIEF DESCRIPTION OF THE FIGURES

FIG. 1A is a schematic representation of an antisense binding complex in which an antisense binding probe described herein is hybridized to an antisense oligonucleotide sequence.

FIG. 1B is a schematic representation of a sense binding complex in which a sense binding probe described herein is hybridized to a sense oligonucleotide sequence.

FIG. 2A is a schematic representation of an antisense binding complex formed between an antisense binding strand of an oligonucleotide duplex and a “short” antisense binding probe.

FIG. 2B is a schematic representation of a sense binding complex formed between a sense binding strand of an oligonucleotide duplex and a “short” sense binding probe.

FIG. 2C is a schematic representation of showing a “non-productive” binding complex formed between a “short” sense binding probe and a “short” antisense binding probe.

FIG. 3A is a schematic representation of an antisense binding complex formed between an antisense binding strand of an oligonucleotide duplex and a “full-length” antisense binding probe.

FIG. 3B is a schematic representation of a sense binding complex formed between a sense binding strand of an oligonucleotide duplex and a “full-length” sense binding probe.

FIG. 3C is a schematic representation of showing a “non-productive” binding complex formed between a “full-length” sense binding probe and a “full-length” antisense binding probe.

FIG. 4A is a schematic of an oligonucleotide tag of a probe hybridized to a capture oligonucleotide immobilized on a support surface, wherein the probe is part of an antisense or sense complex with a single-stranded overhang.

FIG. 4B is a schematic of an oligonucleotide tag of a probe hybridized to a capture oligonucleotide immobilized on a support surface, wherein the probe is part of an antisense or sense complex that does not have a single-stranded overhang.

FIG. 4C is a schematic of an oligonucleotide tag of a probe hybridized to a capture oligonucleotide immobilized on a support surface, wherein the probe is not part of an antisense or sense complex.

FIG. 4D is a schematic of an oligonucleotide tag of a probe hybridized to a capture oligonucleotide immobilized on as support surface, wherein the probe is part of a probe-probe complex that includes a single-stranded overhang.

FIG. 4E is a schematic of an oligonucleotide tag of a probe hybridized to a capture oligonucleotide immobilized on as support surface, wherein the probe is part of a probe-probe complex that does not include a single-stranded overhang.

FIG. 5A is a table showing siRNA sense strand (SS) and antisense strand (AS) detection using an RNase Protection Assay at 37° C. with or without Lysis Buffer, and hybridization treatment at 37° C.

FIG. 5B is a table showing siRNA SS and AS detection using an RNase Protection Assay 37° C. with or without Lysis Buffer, and hybridization treatment at 30° C.

FIG. 5C is a table showing siRNA SS and AS detection using an RNase Protection Assay at 30° C. with or without Lysis Buffer, and hybridization treatment at 30° C.

FIG. 6A is a table showing siRNA sense and antisense strand detection using an RNase Protection Assay at 30° C., 30 min incubation, with or without Lysis Buffer, and hybridization treatment at 30° C.

FIG. 6B is a table showing siRNA sense and antisense strand detection using an RNase Protection Assay at 30° C., 1 h incubation, with or without Lysis Buffer, and hybridization treatment at 30° C.

FIG. 7A is a graph showing siRNA SS and AS strand singleplex detection as a function of probe length.

FIG. 7B is a graph showing siRNA SS and AS strand multiplex detection as a function of probe length.

FIG. 8 is a table showing siRNA sense strand multiplex detection reproducibility in mouse plasma.

FIG. 9 is a table showing siRNA antisense strand multiplex detection reproducibility in mouse plasma.

FIG. 10 is a table showing siRNA sense strand multiplex detection spike recovery in mouse plasma.

FIG. 11 is a table showing siRNA antisense strand multiplex detection spike recovery in mouse plasma.

FIG. 12 is a table showing siRNA sense strand multiplex detection ULOQ (upper limit of quantitation) and LLOQ (lower limit of quantitation) in mouse plasma.

FIG. 13 is a table showing siRNA antisense strand multiplex detection ULOQ and LLOQ in mouse plasma.

FIG. 14 is a table showing linearity of dilution for both siRNA SS and AS strands, in mouse plasma using an siRNA multiplex detection assay.

FIG. 15A is a table showing spike recovery in mouse liver lysate using siRNA multiplex detection assay.

FIG. 15B is a table showing spike recovery of siRNA SS and AS strands in mouse brain lysate using siRNA multiplex detection assay.

### DETAILED DESCRIPTION OF THE INVENTION

### A. Definitions

Unless otherwise defined, scientific and technical terms used herein shall have the meanings that are commonly understood by those of ordinary skill in the art. Further, unless otherwise required by context, singular terms shall include pluralities and plural terms shall include the singular, for example, “a” or “an”, include pluralities, e.g., “one or more” or “at least one” and the term “or” can mean “and/or”, unless stated otherwise. The terms “including,” “includes” and “included”, are not limiting. Ranges provided herein, of any type, include all values within a particular range described and values about an endpoint for a particular range. As used herein, ranges expressed using the word “between” are inclusive of the range endpoints. Thus, for example, a range of between 50° C. and 70° C. includes 50° C. to 70° C., i.e., it includes the endpoints of 50° C. and 70° C.

As used herein, the term “about” is used to modify, for example, the quantity of an ingredient in a composition, concentration, volume, process temperature, process time, yield, flow rate, pressure, and ranges thereof, employed in describing the disclosure. The term “about” refers to variation in the numerical quantity that can occur, for example, through typical measuring and handling procedures used for making compounds, compositions, concentrates or formulations; through inadvertent error in these procedures; through differences in the manufacture, source, or purity of starting materials or ingredients used to carry out the methods, and other similar considerations. The term “about” also encompasses amounts that differ due to aging of a formulation with a particular initial concentration or mixture, and amounts that differ due to mixing or processing a formulation with a particular initial concentration or mixture. Where modified by the term “about,” the claims appended hereto include such equivalents.

Generally, nomenclatures used in connection with, and techniques of, cell and tissue culture, molecular biology, and protein and oligo- or polynucleotide chemistry and hybridization described herein are those well-known and commonly used in the art. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “nucleotide” refers to a monomeric unit that includes a nucleobase, a sugar, and one or more internucleotidic linkages. As used herein, the term nucleotide includes naturally occurring nucleotides and modified nucleotides. Naturally occurring nucleotides include guanine, (G), adenine, (A), cytosine, (C), thymine, (T), and uracil (U) as well as naturally occurring base analogs. In deoxyribonucleic acid (DNA), the sugar is deoxyribose. In ribonucleic acid (RNA), the sugar is ribose. The term “modified nucleotide” refers to a nucleotide that includes a modification at a nucleobase, a sugar or an internucleotidic linkage, wherein the modified nucleotide remains capable of base-pairing to a complementary naturally occurring or modified nucleotide. The term “polynucleotide” refers to polymer of two or more nucleotides covalently linked to each other by an internucleosidic linkage.

As used herein the term “oligonucleotide” refers to a short polymer that includes two or more nucleotides, generally from about 5 to about 100 nucleotides covalently linked by internucleosidic linkages. In one aspect, the oligonucleotide is a polymer that is from about 5, 6, 7, 8, 9, 10, 11, 12, 13, 14, 15, 20, 25 or 30 nucleotides and up to about 30, 35, 40, 45, 50 or 100 nucleotides in length, or from about 8 to about 50 nucleotides in length, about 10 to about 40 nucleotides in length, about 12 to about 30 nucleotides in length, or about 18 to about 30 nucleotides in length. As used herein, the term oligonucleotide can refer to a single-stranded oligonucleotide or a double stranded oligonucleotide, or the individual oligonucleotide strands of a double-stranded oligonucleotide. In one aspect, the term “oligonucleotide” refers to a double-stranded oligonucleotide therapeutic.

An “oligonucleotide therapeutic” is an oligonucleotide that includes at least one strand that is at least partially complementary to and can hybridize to a target nucleic acid. In one aspect, the oligonucleotide therapeutic includes a sense strand and an antisense strand. In one aspect, the oligonucleotide therapeutic can hybridize to the target nucleic acid and modulate expression or an amount of the target nucleic acid. The term “modulates” can include increasing or decreasing expression or an amount of the target nucleic acid. The term “expression” refers to the process by which information in a gene is used to produce a protein and includes, but is not limited to, transcription, splicing, post-transcriptional modification, and translation. In one aspect, the oligonucleotide therapeutic increases expression or an amount of a target nucleic acid. In one aspect, the oligonucleotide therapeutic decreases expression or an amount of a target nucleic acid. In one aspect, the oligonucleotide is chemically synthesized and purified or isolated. In one aspect, the oligonucleotide is made by solid phase chemical synthesis. Methods for making oligonucleotides, including, for example, sense and antisense oligonucleotides, probes, tags or capture oligonucleotides as described herein, are known.

As used herein the terms “nucleic acid”, “polynucleotide”, “oligonucleotide” are used interchangeably and refer to naturally-occurring or synthetic polymeric forms of nucleotides. The oligonucleotides and nucleic acid molecules of the present disclosure may be formed from naturally occurring nucleotides, for example forming deoxyribonucleic acid (DNA) or ribonucleic acid (RNA) molecules. Alternatively, the naturally occurring oligonucleotides may include structural modifications to alter their properties, such as in peptide nucleic acids (PNA) or in locked nucleic acids (LNA). The solid phase synthesis of oligonucleotides and nucleic acid molecules with naturally occurring or artificial bases is well known in the art. The terms should be understood to include equivalents, analogs of either RNA or DNA made from nucleotide analogs and as applicable to the embodiment being described, single-stranded or double-stranded polynucleotides. Nucleotides useful in the disclosure include, for example, naturally-occurring nucleotides (for example, ribonucleotides or deoxyribonucleotides), or natural or synthetic modifications of nucleotides, or artificial bases. As used herein, the term monomer refers to a member of a set of small molecules which are and can be joined together to from an oligomer, a polymer or a compound composed of two or more members. The particular ordering of monomers within a polymer is referred to herein as the “sequence” of the polymer. As used herein the terms “monomer”, “nucleotide”, and “nucleoside” are used interchangeably

In the context of the present disclosure, the term “oligonucleotide” refers to polymeric structures which are capable of hybridizing to at least a region of a “template” sequence of an mRNA, a small non-coding RNA molecule or a target of small non-coding RNAs, or polymeric structures which are capable of mimicking small non-coding RNAs. In one aspect, the term “oligonucleotides” includes, but is not limited to, compounds comprising oligonucleosides, oligonucleotide analogs, oligonucleotide mimetics and combinations of these. In one aspect, oligonucleotides also include, but are not limited to, antisense oligomeric compounds, antisense oligonucleotides, small-interfering RNAs (siRNAs), microRNAs (miRNAs), piwi-interfering (piRNAs), alternate splicers, primers, probes and other compounds that hybridize to at least a portion of the template nucleic acid. Oligonucleotides are routinely prepared linearly but can be joined or otherwise prepared to be circular and may also include branching. Separate oligonucleotides can hybridize to form double stranded compounds that can be blunt-ended or may include overhangs on one or both termini. In one aspect, an oligonucleotide comprises a backbone of linked monomeric subunits where each linked monomeric subunit is directly or indirectly attached to a heterocyclic base moiety. The linkages joining the monomeric subunits, the sugar moieties or sugar surrogates and the heterocyclic base moieties can be independently modified giving rise to a plurality of motifs for the resulting oligonucleotides including hemimers, gapmers and chimeras. Modified oligonucleotides are often preferred over native forms because of desirable properties such as, for example, enhanced cellular uptake, enhanced affinity for nucleic acid and increased stability in the presence of nucleases. As used herein, the term “modification” includes substitution and/or any change from a starting or natural oligomeric compound, such as an oligonucleotide.

As used herein, “base-pairing” refers to specific hydrogen bonding between purines and pyrimidines that leads to the formation of a double-stranded oligonucleotide. In DNA, adenine (A) pairs with thymine (T), and guanine (G) pairs with cytosine (C). In RNA, adenine (A) pairs with uracil (U), and guanine (G) pairs with cytosine (C). While not limited to any particular mechanism, the most common mechanism of base-pairing involves hydrogen bonding, which may be Watson-Crick, Hoogsteen or reversed Hoogsteen hydrogen bonding, between complementary nucleobases.

The term “chimeric” refers to a compound having at least two chemically distinct regions. In one aspect, each region has a plurality of subunits. As used herein, the term “chimeric probe” includes linked single-stranded DNA and/or RNA derived from two or more biological sources.

“Complementary” refers to nucleic acid molecules or oligonucleotides that interact by the formation of hydrogen bonds, for example, according to the Watson-Crick, Hoogsteen or reversed Hoogsteen hydrogen bonding base-pairing models. Hybridization can occur between two complementary DNA molecules (DNA-DNA hybridization), two RNA molecules (RNA-RNA hybridization), or between complementary DNA and RNA molecules (DNA-RNA hybridization). When used in connection with nucleotides, the term “complementary” refers to a pair of nucleotides, for example, that includes a purine and a pyrimidine, which are capable of base-pairing with each other. The complementary pair of nucleotides can include a pair of naturally occurring nucleotides, a pair of modified nucleotides or a pair that includes a naturally occurring nucleotide and a modified nucleotide. When used in connection with an oligonucleotide, the term “complementary” means that nucleotides of one oligonucleotide or a portion thereof are capable of hydrogen bonding nucleotides of another oligonucleotide or portion thereof other when the complementary nucleotides are aligned. Hybridization can occur between a short nucleotide sequence that is complementary to a portion of a longer nucleotide sequence. Hybridization can occur between sequences that do not have 100% “sequence complementarity” (i.e., sequences where less than 100% of the nucleotides align based on a base-pairing model such as the Watson-Crick, Hoogsteen or reversed Hoogsteen hydrogen bonding base-pairing models), although sequences having less sequence complementarity are less stable and less likely hybridize than sequences having greater sequence complementarity. In one aspect, the nucleotides of the complementary sequences have 100% sequence complementarity (i.e., each nucleotide of one oligonucleotides sequence or region can hydrogen bond with each nucleotide of a second oligonucleotide strand or region) based on the Watson-Crick model. In another aspect, the nucleotides of the complementary sequences have at least about 90%, about 95%, about 96%, about 97%, about 98% or about 99% sequence complementarity based on the Watson-Crick model. In one aspect, “substantial complementarity” refers to sequences that are partially complementary and are able to hybridize under physiologically relevant conditions. In one aspect, “substantial complementarity” refers to sequences that are partially complementary and are able to hybridize under stringent hybridization conditions. In one aspect, complementarity refers to the complementarity between two oligonucleotides of a double-stranded oligonucleotide therapeutic. In one aspect, complementarity refers to the complementarity between a single stranded oligonucleotide therapeutic and a single-stranded oligonucleotide probe. In one aspect, complementarity refers to the complementarity between a single stranded oligonucleotide tag and a single stranded capture oligonucleotide. In one aspect, complementarity refers to the complementarity between a single stranded oligonucleotide and a chimeric probe.

Whether or not two complementary sequences hybridize can depend on the stringency of the hybridization conditions, which can vary depending on conditions such as temperature, solvent, ionic strength and other parameters. The stringency of the hybridization conditions can be selected to provide selective formation or maintenance of a desired hybridization product of two complementary nucleic acid sequences, in the presence of other potentially cross-reacting or interfering sequences. Stringent conditions are sequence-dependent—typically longer complementary sequences specifically hybridize at higher temperatures than shorter complementary sequences. Generally, stringent hybridization conditions are between about 5° C. to about 10° C. lower than the thermal melting point (Tₘ) (i.e., the temperature at which 50% of the sequences hybridize to a substantially complementary sequence) for a specific nucleotide sequence at a defined ionic strength, concentration of chemical denaturants, pH and concentration of the hybridization partners. Generally, nucleotide sequences having a higher percentage of G and C bases hybridize under more stringent conditions than nucleotide sequences having a lower percentage of G and C bases. Generally, stringency can be increased by increasing temperature, increasing pH, decreasing ionic strength, or increasing the concentration of chemical nucleic acid denaturants (such as formamide, dimethylformamide, dimethylsulfoxide, ethylene glycol, propylene glycol and ethylene carbonate). Stringent hybridization conditions typically include salt concentrations of less than about 1 M, about 500 mM, or about 200 mM; hybridization temperatures above about 20° C., about 30° C., about 40° C., about 60° C. or about 80° C.; and chemical denaturant concentrations above about 10%, about 20%, about 30%, about 40% or about 50%. Because many factors can affect the stringency of hybridization, the combination of parameters may be more significant than the absolute value of any parameter alone.

In one aspect, complementarity refers to the complementarity between an oligonucleotide and a target nucleic acid sequence. In one aspect, the target nucleic acid includes DNA or RNA. In one aspect, the target RNA includes siRNA, mRNA, pre-mRNA, non-coding RNA, pri-microRNA, pre-microRNA, mature microRNA, or promoter-directed RNA. In one aspect, the target nucleic acid is a cellular gene or mRNA transcribed from the gene whose expression is associated with a particular disorder or disease. In one aspect, the target nucleic acid is a nucleic acid molecule from an infectious agent. In one aspect, the target nucleic acid is a viral or bacterial nucleic acid.

As used herein, “hybridize,” “hybridizing” or “hybridization” refers to base-pairing between two complementary oligonucleotides. In one aspect, a single-stranded oligonucleotide strand of a double-stranded oligonucleotide therapeutic can hybridize to a target nucleic acid. In one aspect, two single-stranded oligonucleotide strands of a double-stranded oligonucleotide hybridize to each other. In one aspect, an oligonucleotide probe hybridizes to a single-stranded oligonucleotide strand of a double-stranded oligonucleotide. In one aspect, a single-stranded oligonucleotide tag hybridizes to a single-stranded capture oligonucleotide. “Specifically hybridize” refers to hybridization between two complementary oligonucleotides that occurs with greater affinity and without significant cross-hybridization with other oligonucleotides in a sample. In one aspect, the complementary oligonucleotides specifically hybridize under physiologically relevant conditions, such as the conditions found in the cytoplasm of a cell. In another aspect, the complementary oligonucleotides specifically hybridize under stringent hybridization conditions.

As used herein, “oligonucleotide duplex” refers to a double-stranded oligonucleotide that is formed by hybridization of two single-stranded oligonucleotides that are at least partially complementary to one another and hybridize to one another via base-pairing between complementary nucleobases. In one aspect, at least one strand of the oligonucleotide duplex includes DNA. In one aspect, at least one strand of the oligonucleotide duplex includes RNA. In one aspect, both strands of the oligonucleotide duplex include DNA. In one aspect, both strands of the oligonucleotide duplex include RNA. In one aspect, the double-stranded oligonucleotide is a heteroduplex that includes one strand that is DNA and one strand that is RNA. In one aspect, the sugar-phosphate backbones of the two oligonucleotide strands of the oligonucleotide duplex are oriented in opposite directions (i.e., one strand runs 5′ to 3′ and the other 3′ to 5′), which is referred to as “antiparallel”. In one aspect, the two oligonucleotide strands of the oligonucleotide duplex are the same length as one another such that the oligonucleotide duplex is double-stranded over its entire length, i.e., the oligonucleotide duplex has blunt ends. In one aspect, the two oligonucleotide strands of the oligonucleotide duplex are the same length as one another but align in such a manner that the oligonucleotide duplex is not double-stranded over its entire length, i.e., the oligonucleotide duplex has a single stranded 3′ overhang or a single stranded 5′ overhang at both ends of the duplex. In one aspect, the two oligonucleotide strands of the oligonucleotide duplex are a different length from each other such that the oligonucleotide duplex is not double-stranded over its entire length, i.e., the oligonucleotide duplex has a single stranded 3′ overhang or a single stranded 5′ overhang at one or both ends of the oligonucleotide duplex. In one aspect, the single stranded overhang is from about 1 to about 5, about 1 to about 4, about 1 to about 3, or about 1 to about 2 nucleotides. In one aspect, the oligonucleotide duplex has one blunt end and one end that includes a single stranded overhang. When used in connection with an oligonucleotide, the term “length” refers to the number of nucleotide residues in the polymeric backbone of a single-stranded oligonucleotide.

As used herein, the term “overhang” refers to a double-stranded oligonucleotide in which at least one end of one strand is longer than the corresponding end of the other strand. In one aspect, the single-stranded overhang is located at the 3′-terminus of one or both strands of the double stranded oligonucleotide. In one aspect, the single-stranded overhang is located at the 5′-terminus of one or both strands of the double stranded oligonucleotide. In one aspect, the single-stranded overhang includes from about 1 to about 5 nucleotides, from about 1 to about 4 nucleotides, from about 1 to about 3 nucleotides, or from about 1 to about 2 nucleotides. In one aspect, one end of the double-stranded oligonucleotide is blunt and the other end includes a 3′ or a 5′ overhang. In one aspect, both ends of the double-stranded oligonucleotide include a single stranded overhang.

The term “antisense” refers to an oligonucleotide with a nucleic acid sequence that is inverted relative to the orientation necessary for transcription of a target nucleic acid, such that the antisense oligonucleotide and can hybridize to the target nucleic acid, for example, through Watson-Crick base-pairing. The “sense strand” of the oligonucleotide duplex is complementary to the antisense strand and is therefore “sense” to at least part of the target nucleic acid. In one aspect, the antisense and sense strands of the oligonucleotide duplex are at least about 90%, about 95%, about 96%, about 97%, about 98%, about 99% or about 100% complementary to one another.

A single stranded oligonucleotide has “direction” or “directionality” because adjacent nucleotides are joined by an internucleosidic linkage, such as a phosphodiester bond between their 5′ and 3′ carbons atoms, such that the terminal 5′ and 3′ carbons are exposed at either end of the oligonucleotide, which can be referred to as the 5′-(phosphoryl) and 3′-(hydroxyl) ends of the molecule.

The term “identical” means that an oligonucleotide sequences include identical nucleic acid bases at the same positions over a comparison window. The term “% sequence identity” can be determined by comparing two aligned sequences over a window of comparison, determining the number of positions at which the identical nucleic acid base occurs in both sequences to yield the number of matched positions, dividing the number of matched positions by the total number of positions in the comparison window, and multiplying the result by 100 to yield the percentage of sequence identity. The comparison window can include a full-length sequence or may be a subpart of a larger sequence. Various methods and algorithms are known for determining the percent identity between two or sequences, including, but not limited MEGALIGN (DNASTAR, Inc. Madison, Wis.), FASTA, BLAST, or ENTREZ.

In one aspect, a nucleotide of an oligonucleotide described herein includes a structural analog with a non-naturally occurring chemical structures that can also participate in hybridization reactions. In one example, a nucleotide or nucleic acid may include a chemical modification that links it to a label or provides a reactive functional group that can be linked to a label, for example, through the use of amine or thiol-modified nucleotide bases, phosphates or sugars. The term “reactive functional group” refers to an atom or associated group of atoms that can undergo a further chemical reaction, for example, to form a covalent bond with another functional group. Examples of reactive functional groups include, but are not limited to, amino, thiol, hydroxy, and carbonyl groups. In one aspect, the reactive functional group includes a thiol group. Labels that can be linked to nucleotides or nucleic acids through these chemical modifications include, but are not limited to, detectable moieties such as biotin, haptens, fluorophores, and electrochemiluminescent (ECL) labels.

The term “modified oligonucleotide” refers to an oligonucleotide that includes at least one nucleoside modification, for example, a sugar modification or a nucleobase modification; or an internucleoside linkage modification. In one aspect, the modified oligonucleotide includes one or more modifications that include, but are not limited to, phosphodiester (PO); phosphorothioate (PS); 2′O-methyl (2′OMe); 2′O-methoxyethyl (MOE); phosphorothioate constrained ethyl (cEt); peptide nucleic acid (PNA); phosphoroamidate morpholino (PMO); locked nucleic acid (LNA); 2′-deoxy-2′-fluoro (2′-F); or a combination thereof. “Locked nucleic acid nucleoside” or “LNA” refers a nucleoside that includes a bicyclic sugar moiety with a 4′-CH₂—O-2′bridge. “Phosphorothioate” refers to an internucleotide linkage in which one of the non-bridging oxygens is replaced by sulfur.

The term “conjugate” refers to an atom or group of atoms that is directly or indirectly attached to an oligonucleotide. In one aspect, the conjugate is connected to the oligonucleotide through a stable linker or a cleavable linker. In one aspect, the conjugate modifies one or more properties of the oligonucleotide to which it is attached, including, but not limited to pharmacodynamics, pharmacokinetic, binding, absorption, cellular distribution, cellular uptake, charge or clearance properties. Examples of conjugates include, but are not limited to, polyethylene glycol (PEG), N-acetylegalactosamine (GalNAc), cell penetrating peptides (CPP), vitamin E (also known as α-tocopherol), aptamers, antibodies, cholesterol or cholesterol derivatives, squalene, fatty acids, nucleolipids, and spherical nucleic acids.

The term “nuclease” refers to an enzyme, for example, a hydrolase, that can cleave the backbone of an oligonucleotide polymer. In one aspect, the nuclease is a phosphodiesterase that cleaves a phosphodiester linkage in the backbone of an oligonucleotide. “Ribonuclease” or “RNase” refers to an enzyme that preferentially cleave ribonucleic acid (RNA). “Deoxyribonuclease” or “DNase” refers to an enzyme that preferentially cleaves deoxyribonucleic acid (DNA). In one aspect, the nuclease is a “single-strand specific nuclease” that preferentially cleaves single-stranded oligonucleotides or single-stranded regions of a double-stranded oligonucleotide. A single-strand specific RNase is an enzyme that preferentially cleaves single-stranded RNA. A single-strand specific DNase is an enzyme that preferentially cleaves single-stranded DNA.

“Target nucleic acid” refers to a nucleic acid of interest with a known sequence to which an oligonucleotide is designed to hybridize. In one aspect, the target nucleic acid is a nucleic acid with a known sequence to which an oligonucleotide therapeutic is designed to hybridize. In one aspect, the target nucleic acid is a sequence found in the DNA or RNA of a prokaryotic or eukaryotic organism. In one aspect, the target nucleic acid includes miRNA, therapeutic RNA, mRNA, an RNA virus, or a combination thereof. In one aspect, hybridization of the oligonucleotide to the target nucleic acid in a cell alters activity of a gene expressed by the cell. In one aspect, hybridization of the oligonucleotide to the target nucleic acid increases activity of a gene. In one aspect, hybridization of the antisense oligonucleotide to the target nucleic acid decreases activity of a gene.

In one aspect, the target nucleic acid includes 16S ribosomal DNA (16S rDNA) or 16 ribosomal RNA (16S rRNA). 16s rRNA is the ribosomal RNA component of the small subunit of ribosomes of prokaryotes responsible for the essential process of converting genetic messages to functional cell components via the translation of mRNA to proteins. The gene 16s rDNA encodes the 16S rRNA sequence. The 16S rRNA gene is conserved in bacteria, and contain hypervariable regions that can provide species-specific signature sequences and is widely used in identification of bacteria and phylogenetic, identification, classification and quantitation studies.

In one aspect, the target nucleic acid is an siRNA. In one aspect, the siRNA comprises short double-stranded RNA that is about 17 nucleotides to about 29 nucleotides in length, or about 19 to about 25 nucleotides in length. siRNAs typically comprise a sense RNA strand and a complementary antisense RNA strand annealed together by standard Watson-Crick base-pairing interactions (hereinafter “base-paired”). In one aspect, the sense and antisense strands of the siRNA comprise two complementary, single-stranded RNA molecules or comprise a single molecule in which two complementary portions are base-paired and are covalently linked by a single-stranded “hairpin” area. In one aspect, the siRNA also contains alterations, substitutions, or modifications of one or more ribonucleotide bases.

RNA interference (RNAi) is a biological process in which double-stranded RNA molecules, like siRNA, are involved in sequence-specific suppression of gene expression through translational or transcriptional repression. Sense and/or antisense strands of the disclosure that comprise nucleic acid sequences substantially identical to a template sequence are characterized in that such sense and/or antisense strands induce RNAi-mediated degradation of nucleic acid containing the template sequence. In one aspect, an siRNA of the disclosure comprises a sense and/or antisense strand comprising nucleic acid sequences that differ from a template sequence by one, two or three or more nucleotides, as long as RNAi-mediated degradation of the template nucleic acid is induced by the siRNA. The term “subject” or “patient” refers to an organism to which an oligonucleotide composition is administered for experimental, diagnostic, prophylactic or therapeutic purposes and includes, but is not limited to animals, for example, mammals such as mice, rats, rabbits, non-human primates, and humans; insects; worms; and plants. In one aspect, a subject may be suffering from or susceptible to a disease or disorder.

Small interfering RNA (siRNA) molecules play a key role in gene regulation and therapeutic applications. Currently, there are at least five FDA-approved siRNA drugs, with many more in various stages of the clinical trial pipeline, making their precise detection of paramount importance in preclinical research and translational medicine. Measuring both strands of therapeutic siRNA is essential for optimizing efficacy, ensuring specificity, and addressing safety concerns, including off-target effects and immunostimulation. Most pharmacokinetic assays for the detection of duplex siRNA rely on low-sensitivity formats like LC-MS or singleplex measurements of only the antisense strand (AS) and a separate assay for the sense strand (SS). We sought to develop an assay that can measure both strands of a duplex siRNA, in a single well, with high sensitivity.

The disclosure provides a rapid and efficient approach for the multiplex detection of both strands of duplex siRNA using an ultrasensitive electrochemiluminescence (ECL) assay. Simultaneous detection of both strands of duplex siRNA in a single well is challenging using existing technologies, since the strands share a high degree of sequence complementarity, leading to cross-hybridization between capture probes and high false positive signals. The disclosure addresses this using specially-designed capture probes, tailored to target the SS and AS strands of siRNA, while eliminating the risk of cross-hybridization. In embodiments, these capture probes are shorter than the SS and AS strands by two or more nucleotides. Shortened probes specific for SS or AS hybridize to their target strands with high stringency in a single well, instead of cross-hybridizing to each other. in embodiments, any residual probe-probe interactions are also degraded by the added RNases at the free single-stranded RNA sections not covered by the shortened probes. In embodiments, hybridized probe-analyte complexes are then detected via an RNase protection assay using ECL-based readout. Assay conditions are optimized to achieve reproducible and highly sensitive detection for both siRNA strands.

Using the methods disclosed herein, multiplex detection of duplex siRNA was achieved at remarkably low sub-pM concentrations in a single well. In embodiments, the lower limits of detection (LLODs) were 0.15 pM for AS and 0.28 pM for SS strands respectively. In embodiments, experimentally-determined lower limits of quantitation (LLOQs) were 0.8 pM for both SS and AS strands.

The versatility and robustness of this detection strategy in complex biological matrices such as mouse plasma, brain, and liver is provided by the these methods. In embodiments, recoveries of high-, mid- and low-spikes in mouse brain and liver tissue lysates ranged between 83%-118%. In plasma, for example, the siRNA detection methods of the disclosure show excellent linearity of dilution throughout the dynamic range.

The siRNA detection methods of the disclosure, which in embodiments, combine siRNA strand specificity with highly sensitive ECL detection, represent a significant advancement in siRNA analysis techniques for pharmacokinetic studies.

As used herein, the term “human microbiome” refers to the collection of all microbes, such as bacteria, fungi, viruses, and their genes, that naturally live on and within the human body. In one aspect, the microbes of the human microbiome live on or within human organs, tissues and bodily fluids including the skin, mammary glands, nasal passages, seminal fluid, uterus, ovarian follicles, lung, saliva, oral mucosa, conjunctiva, biliary tract, and gastrointestinal tract. In one aspect, the human microbiome consists of microbes that are commensal and co-exist without harming humans. In one aspect, the human microbiome consists of microbes that are helpful to the human body. In once aspect, the human microbiome consists of microbes that are harmful to the human body. In one aspect, the human microbiome consists of groups of microbes that are helpful and harmful to the human body. In one aspect, the human microbiome consists of microbes that are symbiotic wherein both the human body and microbiota benefit.

“Probe” refers to a reagent that includes a single stranded oligonucleotide sequence that is capable of hybridizing to a single-strand of an oligonucleotide duplex. In one aspect, the probe includes a single stranded oligonucleotide sequence that is capable of hybridizing to a sense or an antisense strand of an oligonucleotide duplex. A “sense probe” is an oligonucleotide that includes a single stranded oligonucleotide sequence that is capable of hybridizing to a sense strand of an oligonucleotide duplex. An “antisense probe” is an oligonucleotide that includes a single stranded oligonucleotide sequence that is capable of hybridizing to an antisense strand of an oligonucleotide duplex. In one aspect, the probe includes a single stranded oligonucleotide sequence that is complementary or substantially complementary to a sense or an antisense strand of an oligonucleotide duplex. In one aspect, the probe includes an oligonucleotide tag (which can be referred to as a targeting sequence) that is complementary to sequence of a capture oligonucleotide. Probes can include DNA or RNA or a combination of DNA and RNA sequences and can include one or more modified nucleotides or modified internucleotidic linkages. Probes can be prepared by any suitable method known in the art, including, but not limited to, chemical or enzymatic synthesis.

“Linker” refers to one or more atoms that join one chemical moiety to another chemical moiety. In one aspect, a linker joins a reactive functional group or label to an oligonucleotide. The linker can be a nucleotide or non-nucleotide compound that includes one or more atoms, for example, from about 2, about 3, about 4, about 5, about 6, about 7, about 8, about 9 or about 10 atoms to about 11, about 12, about 13, about 14, about 15, about 16, about 17, about 18, about 19, or about 20 atoms and can include atoms such as carbon, oxygen, sulfur, nitrogen and phosphorus and combinations thereof. Examples of linkers include low molecular weight groups such as amide, ester, carbonate and ether groups, as well as higher molecular weight linking groups such as polyethylene glycol (PEG) and alkyl chains. Linkers may include one or more atoms, units, or molecules.

“Label” refers to a chemical group or moiety that has a detectable physical property or is capable of causing a chemical group or moiety to exhibit a detectable physical property, including, for example, an enzyme that catalyzes conversion of a substrate into a detectable product. A label can be detected by spectroscopic, photochemical, biochemical, immunochemical, electrical, optical, chemical, or other methods. Examples of labels include, but are not limited to, radioisotopes, enzymes, substrates, fluorescent molecules, chemiluminescent moieties, electrochemiluminescent moieties, magnetic particles, and bioluminescent moieties. In another aspect, the label is a compound that is a member of a binding pair, in which a first member of the binding pair (which can be referred to as a “primary binding reagent”) is attached to a substrate, for example, an oligonucleotide, and the other member of the binding pair (which can be referred to as a “secondary binding reagent”) has a detectable physical property or is attached to a moiety with a detectable physical property. Non-limiting examples of binding pairs include biotin and streptavidin, or avidin; complementary oligonucleotides; hapten and hapten binding partner; and antibody/antigen binding pairs.

As used herein, “concurrently” when used in connection with method steps described herein, refers to a method in which the steps are performed substantially at the same time, i.e., in which the execution of at least a portion of one method step overlaps in time with the execution of a portion of another method step. “Concurrently” does not require exact simultaneous activity, i.e., it is not required that all method steps begin or end at the same time. In one aspect, “concurrently” can mean that all reagents necessary for the method steps are combined in the same reaction mixture such that the reactions occur in the same reaction volume or during the same incubation period.

As used herein, “sequentially” when used in connection with method steps described herein, refers to a method in which the steps are performed at different points in time, for example, in which separate events occur in the practice of the method. In one aspect, the sequential steps are performed during separate incubation periods. In one aspect, the sequential steps are performed in different reaction mixtures. In one aspect, the sequential method steps are performed at a different time. In one aspect, the sequential method steps are performed at a different time, but in the same reaction cell or on the same surface.

“Capture oligonucleotide” refers to an oligonucleotide reagent that can be immobilized on a support surface and is designed to hybridize to (and, therefore, to capture on the surface) a complementary oligonucleotide tag. In one aspect, the capture oligonucleotide is a single stranded sequence that can selectively hybridize, for example, under stringent hybridization conditions, with a single stranded oligonucleotide tag present on an oligonucleotide probe. Capture oligonucleotides may be provided in solid form (e.g., lyophilized), in solution, or immobilized to a support surface, e.g., on particles (e.g., microparticles, beads) or in arrays.

“Detection” can refer to detecting or quantifying the presence of a substance, such as an oligonucleotide, based on the presence or absence of a label. In one aspect, “detecting” refers to a process in which the presence or absence of a substance, such as an oligonucleotide is determined. In one aspect, “quantifying” refers to a process when an amount of a substance, such as an oligonucleotide, is determined.

“Corresponding” can be used to refer to the relationship between a capture oligonucleotide and an oligonucleotide tag, wherein the oligonucleotide tag is designed to specifically bind to a particular capture oligonucleotide sequence under stringent hybridization conditions. In one aspect, an oligonucleotide tag specifically binds to its corresponding capture oligonucleotide and does not bind or cross-react with other capture oligonucleotides under stringent conditions. In one aspect, an oligonucleotide tag specifically binds to its corresponding capture oligonucleotide and does not bind or cross-react with other capture oligonucleotides in an array under stringent conditions. In one aspect, the oligonucleotide tag is a single stranded oligonucleotide that has a sequence that is complementary to at least part of a sequence of its “corresponding” capture oligonucleotide. In one aspect, the nucleotides of the “corresponding” oligonucleotide tag and capture oligonucleotide sequences have 100% sequence complementarity based on the Watson-Crick model. In another aspect, the nucleotides of the corresponding sequences have at least about 90%, 95%, 96%, 97%, 98% or 99% sequence complementarity based on the Watson-Crick model.

“Corresponding” can be used to refer to the relationship between a sense binding portion of a sense probe, or an antisense binding portion of an antisense probe and a sense strand or antisense strand, respectively, of an oligonucleotide duplex. In one aspect, a sense binding portion of a sense probe specifically binds to its corresponding sense strand of an oligonucleotide duplex and does not bind or cross-react with the sense strand of other oligonucleotide duplexes in the sample, with the antisense strand of the oligonucleotide duplex, or with an oligonucleotide tag in the sample. In one aspect, an antisense binding portion of an antisense probe specifically binds to its corresponding antisense strand of an oligonucleotide duplex and does not bind or cross-react with the antisense strand of other oligonucleotide duplexes in the sample, with the sense strand of the oligonucleotide duplex, or with an oligonucleotide in the sample. In one aspect, the nucleotides of the “corresponding” sense binding portion or antisense binding portion and sense oligonucleotide or antisense oligonucleotide, respectively, have 100% sequence complementarity based on the Watson-Crick model. In another aspect, the nucleotides of the “corresponding” sense binding portion or antisense binding portion and sense oligonucleotide or antisense oligonucleotide, respectively have at least about 90%, 95%, 96%, 97%, 98% or 99% sequence complementarity based on the Watson-Crick model.

“Cross-react” or “cross-reactive” refers to the ability of an oligonucleotide sequence to hybridize to more than one other oligonucleotide sequence in a sample. In one aspect, the term “cross-react” refers to the ability of a first oligonucleotide sequence to hybridize to a second oligonucleotide sequence in a sample, wherein the second oligonucleotide sequence is not complementary or substantially complementary to the first oligonucleotide sequence. In one aspect, the term “cross-react” or “cross-reactive” refers to the ability of a capture oligonucleotide to hybridize to more than one oligonucleotide tag or more than one tagged target nucleotide sequence in a sample. In one aspect, the cross-reactive capture oligonucleotide hybridizes to one or more oligonucleotide tags in a sample under stringent capture hybridization conditions. “Non-cross-reactive” or “non-cross-reacting” refers to a first oligonucleotide sequence that hybridizes only to a particular oligonucleotide sequence in a sample, for example, the ability of a first oligonucleotide sequence to hybridize only to its corresponding complementary sequence in a sample. In one aspect, the term “non-cross-reactive” refers to the ability of a capture oligonucleotide to hybridize only to one oligonucleotide tag in a sample that include more than one oligonucleotide tag or more than one tagged target nucleotide sequences. In one aspect, the non-cross-reactive capture oligonucleotide hybridizes only to one oligonucleotide tag in a sample under stringent hybridization conditions. In one aspect, non-cross-reactive means that the ratio at which the first oligonucleotide binds to a sequence other than its complementary sequence in a sample is less than 0.05% under stringent hybridization conditions. In one aspect, stringent capture hybridization conditions include a temperature of between 27° C. and 47° C., a formamide concentration between 21% and 41%, a salt concentration between 300 mM and 500 mM and a pH between 7.5 and 8.5. In one aspect, stringent capture hybridization conditions include a temperature less than about 37° C. In one aspect, stringent capture hybridization conditions include a temperature of about 27° C. to about 37° C. In one aspect, stringent capture hybridization conditions include a temperature of about 30° C. or 37° C., a formamide concentration of about 31%, a salt concentration of about 400 mM and a pH of 8.0.

“Array” refers to one or more support surfaces having more than one spatially distinct (i.e., not overlapping) addressable locations, referred to herein as binding domains or array elements. In one aspect, each addressable location includes an assay reagent, including, for example, a capture oligonucleotide.

A “support surface” refers to a surface material onto which, various substances, for example, one or more capture oligonucleotides can be immobilized. A “support surface” can be planar or non-planar. In one aspect, the support surface includes a flat surface. In one aspect, the support surface is a plate with a plurality of wells, i.e., a “multi-well plate.” Multi-well plates can include any number of wells of any size or shape, arranged in any pattern or configuration. In another aspect, the support surface has a curved surface. In one aspect, the support surface is provided by one or more particles, beads or microspheres. The terms particles, beads or microspheres can be used interchangeably unless otherwise indicated. In one aspect, the support surface includes color coded particles, beads or microspheres. In one aspect, the support surface includes an assay module, such as an assay plate, slide, cartridge, bead, or chip. In one aspect, the support surface includes assay flow cells or assay fluidics.

In one aspect, the support surface includes a plurality of addressable locations (which may be referred to as “spots”), for example, as is typical in “gene chip” devices. In another aspect, the array includes a plurality of support surfaces that each have one addressable location, as in “bead array” approaches where each bead in a suspension of beads represents an addressable location (which, for example, may be addressed using flow cytometric or microscopic detection techniques). In another aspect, the array includes a plurality of support surfaces that each have one or more, or two or more addressable locations per surface. The addressable locations on a support surface can be arranged in uniform rows and columns or can form other patterns. The number of addressable locations on the array can vary, for example from less than about 10 to more than about 50, about 100, about 200, about 500, or about 1000. “Multiplexing” refers to the simultaneous analysis of more than one assay target in a single assay.

In the context of analytes measured in an assay, or a reagents used in an assay, the term “plurality” means more than one structurally or functionally different analyte or reagent (e.g., reagent A and reagent B), rather than just more than one copy of the analyte or reagent (e.g., reagent A and another copy of reagent A). For example, the term “plurality of detection reagents” means that more than one structurally or functionally different detection reagent is present in an assay, for example, the different detection reagents each specifically bind a different target analyte and does not describe a situation where there are multiple copies of one reagent. However, use of the term “plurality” in this context does not preclude the possibility that multiple copies are present of any of the plurality of analytes or reagents. For example, a plurality of immobilized targeting reagent complements could refer to immobilized targeting reagent complements that include one or more copies of targeting reagent complement A and one or more copies of targeting reagent complement B. When referring to a plurality of analytes or reagents, the terms “first,” “second,” “third,” etc. or “additional” can be used to distinguish between the unique analytes or reagents. For example, a “first” detection reagent binds to a “first” target analyte and a “second” detection reagent binds to a “second” target analyte or a different portion of the target analyte.

“Unique” is a relative term that depends on the other components present in a composition or mixture. For example, when used in connection with a nucleotide sequence, for example the nucleotide sequence of an analyte binding portion of a probe, the term “unique” means that the nucleotide sequence of one analyte binding portion is different from the nucleotide sequence of the analyte binding portion of the other probes in the composition or mixture. Similarly, when used in connection with a targeting reagent or oligonucleotide tag, the term “unique” means that the nucleotide sequence of the targeting reagent or oligonucleotide tag is different from the nucleotide sequence of other targeting reagents or tag in the composition or mixture. The term “unique” does not preclude the possibility that multiple copies of a “unique” analyte or reagent may be present in an assay or sample.

“Carbon-based” refers to a material that contains elemental carbon (C) as a principal component. Examples of carbon-containing or carbon-based materials include, but are not limited to, carbon, carbon black, graphitic carbon, glassy carbon, carbon nanotubes, carbon fibrils, graphite, carbon fibers and mixtures thereof. Carbon-based materials can include elemental carbon, including, for example, graphite, carbon black or carbon nanotubes. In one aspect, carbon-based materials include conducting carbon-polymer composites, conducting polymers, or conducting particles dispersed in a matrix, for example, carbon inks, carbon pastes, or metal inks. Conducting carbon particles include, for example, carbon fibrils, carbon black, or graphitic carbon, dispersed in a matrix, for example, a polymer matrix such as ethylene vinyl acetate (EVA), polystyrene, polyethylene, polyvinyl alcohol, polyvinyl acetate, polyvinyl chloride or acrylonitrile butadiene styrene (ABS). Such polymer matrices can also include copolymers with more than one type of component monomer which may include monomers selected from vinyl acetate, ethylene, vinyl alcohol, vinyl chloride, acrylonitrile, butadiene, styrene or other monomers.

### B. Overview

Provided herein is a method for detecting or quantifying an oligonucleotide duplex in a sample. In one aspect, a method is provided for detecting or quantifying a first and a second strand of an oligonucleotide duplex in a sample. In one aspect, a method is provided for detecting, or quantifying a sense and an antisense strand of an oligonucleotide duplex in a sample. In one aspect, the method is used for detecting, or quantifying a sense and an antisense strand of a double-stranded oligonucleotide therapeutic.

The method described herein provides a robust and sensitive method for characterizing oligonucleotide therapeutics in a variety of complex biological samples including, for example, biological fluids, including, but not limited to, plasma, serum, whole blood, urine, feces, breast milk, saliva, and amniotic fluid; and tissues or tissue homogenates, including, but not limited to, organs or organ homogenates, such as brain, liver, spleen, heart, lung, and kidney or other tissues, for example, muscle, skin, or bone marrow. In one aspect, the sample is an environmental sample. In one aspect, the sample is a manufacturing process sample.

In one aspect, the method can be used to characterize, for example, pharmacokinetics, biodistribution and cell uptake of a therapeutic oligonucleotide, including, but not limited to, pharmacokinetics (PK), pharmacodynamics (PD), clearance, half-life, peak concentration, exposure-response relationships, biodistribution, tissue targeting, tissue accumulation, tissue bioavailability, or combinations thereof. In one aspect, the method can be used to detect or quantify both strands of an oligonucleotide duplex, for example, to assess the stability of the duplex as well as the pharmacokinetics, biodistribution and cell uptake of the individual strands of an oligonucleotide duplex.

In one aspect, the method or kit is used to identify, detect or quantify one or more nucleotide sequences or variants of a microorganism. In one aspect, the method or kit is used to identify, detect or quantify one or more nucleotide sequences or variants of bacteria, fungi, protozoa or a virus. In one aspect, the method or kit is used to identify, detect or quantify one or more nucleotide sequences of bacteria, fungi, protozoa or a virus that is a component of the human microbiome. In one aspect of the method or kit is used to identify, detect or quantify one or more nucleotide sequences or variants of 16S rRNA or rDNA from bacteria.

In one aspect, the bacteria is Achromobacter, Acidaminococcus, Acinetobacter, Actinomycetales, Aerococcus, Anaerococcus, Aggregatibacter, Aeromonas, Alcaligenes, Anaerobiospirillum, Atopobium, Bacillus, Bacillota, Bacteroides, Bacterionema, Bartonella, Bifidobacterium, Bordetella, Borrelia, Brucella, Burkholderia, Buchnera, Butyriviberio, Campylobacter, Capnocytophaga, Cardiobacterium, Chlamydia, Chlamydophila, Collinsella, Citrobacter, Clostridium, Corynebacterium, Cutibacterium, Dialister, Demodex, Eggerthella, Eikenella, Enterococcus, Enterobacter, Escherichia, Eubacterium, Faecalibacterium, Finegoldia, Firmicutes, Flavobacterium, Francisella, Fusobacterium, Gardnerella, Gordonia, Haemophilus, Helicobacter, Kingella, Klebsiella, Lactobacillus, Legionella, Leptospira, Leptotrichia, Listeria, Megasphaera, Methanobrevibacter, Microbacterium, Micrococcus, Mobiluncus, Morganella, Moraxella, Mycobacterium, Mycoplasma, Neisseria, Peptococcus, Peptoniphilus, Peptostreptococcus, Plesiomonas, Porphyromonas, Prevotella, Propionibacterium, Proteus, Providencia, Pseudomonas, Pseudomonadota, Rickettsia, Roseburia, Rothia, Ruminococcus, Sarcina, Salmonella, Selenomonas, Shigella, Slackia, Sneathia, Spirochaeta, Staphylococcus, Streptobacillus, Streptococcus, Streptomyces, Tannerella, Treponema, Trichophyton, Ureaplasma, Veillonella, Vibrio, Wolinella or Yersinia bacteria.

In one aspect, a method is provided for detecting or quantifying a sense and an antisense strand of an oligonucleotide duplex in a sample. In one aspect, the method includes contacting the sample with a composition that includes a set of probes that includes a sense probe and an antisense probe. In one aspect, the sense probe includes a single stranded oligonucleotide tag that is complementary to at least a portion of a capture oligonucleotide immobilized on a support surface, a sense binding portion capable of hybridizing to a nucleotide sequence of the sense strand of the oligonucleotide duplex, and a label. In one aspect, the antisense probe includes a single stranded oligonucleotide tag that is complementary to at least a portion of a second capture oligonucleotide immobilized on the support surface, an antisense binding portion capable of hybridizing to a nucleotide sequence of the antisense strand of the oligonucleotide duplex, and a label. In one aspect, the sense binding portion of the sense probe has a sense binding length that is shorter than a sense strand length of the sense strand. In one aspect, the antisense binding portion of the antisense strand has an antisense binding length that is shorter than an antisense strand length of the antisense strand.

In one aspect, the method further includes a step of incubating the probes with the sample to form a hybridization mixture. In one aspect, the hybridization mixture includes “productive” or “desirable” hybridization complexes in which the sense probe hybridizes with the sense strand of the oligonucleotide duplex to form a sense complex and the antisense probe hybridizes with the antisense strand of the oligonucleotide duplex to form an antisense complex. In one aspect, the hybridization mixture includes one or more “non-productive” or undesirable hybridization complexes. One example of an unproductive hybridization complex is when the sense probe fails to hybridize to the sense strand of the oligonucleotide or when the antisense probe fails to hybridize to the antisense strand of the oligonucleotide. Another example of an unproductive hybridization complex is when the sense probe and antisense probes hybridize to each other to form a probe-probe complex.

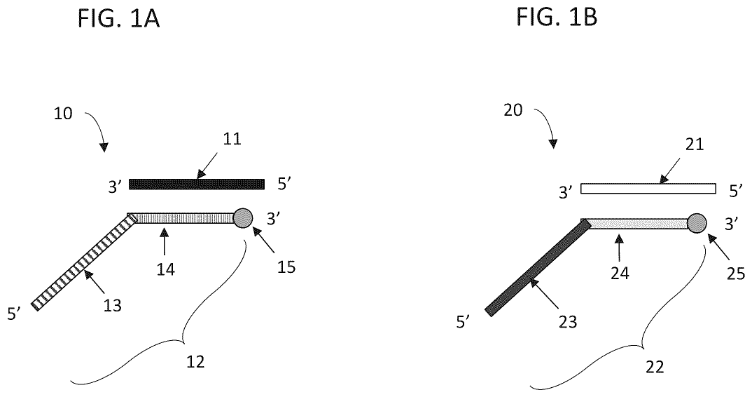

FIG. 1A is a schematic of an antisense complex 10 that includes an antisense strand 11 of an oligonucleotide duplex and an antisense probe 12, wherein the antisense probe 12 includes an oligonucleotide tag 13, an antisense binding portion 14 and a label 15. FIG. 1B is a schematic of a sense complex 20 that includes a sense strand 21 from an oligonucleotide duplex and a sense probe 22, wherein the sense probe 22 includes an oligonucleotide tag 23, a sense binding portion 24 and a label 25.

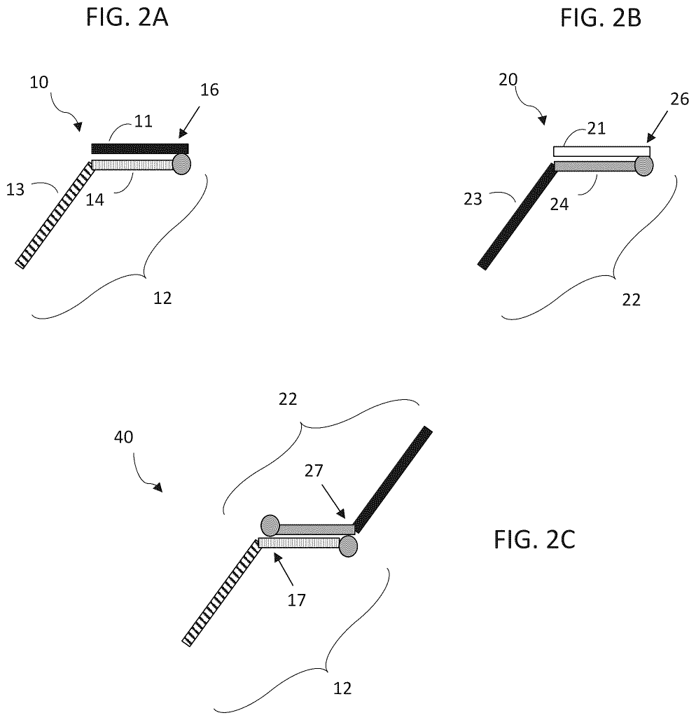

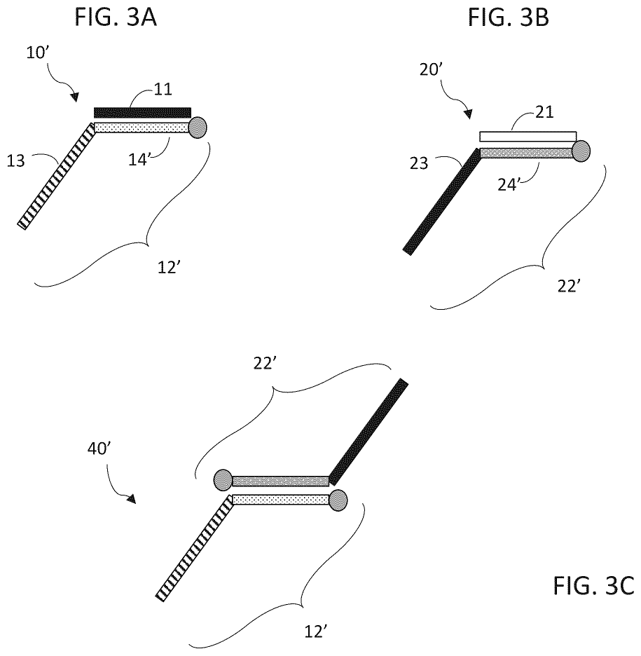

One difficulty arising when trying to detect a sense and an antisense strand of an oligonucleotide duplex is the potential unproductive binding due to probe-probe hybridization. The hybridization complexes that are possible in a hybridization mixture that contain sense and antisense strands of an oligonucleotide duplex and sense and antisense probes are shown in FIG. 2A-2C (using a probe with a short binding portion) and FIG. 3A-3C (using a probe with a full-length binding portion).

FIG. 2A is a schematic of an antisense complex 10 that includes an antisense strand 11 of an oligonucleotide duplex and an antisense probe 12, wherein the antisense probe 12 includes an oligonucleotide tag 13, a “short” antisense binding portion 14 and a label 15. FIG. 2B is a schematic of a sense complex 20 that includes a sense strand 21 from an oligonucleotide duplex and a sense probe 22, wherein the sense probe 22 includes an oligonucleotide tag 23, an “short” sense binding portion 24, and a label 25. As used herein, the term “short” binding portion means that the binding portion of the antisense and sense probes have a length that is shorter than (i.e., includes at least one less nucleotide) the antisense and sense strands of the oligonucleotide duplex, respectively, such that there is a single-stranded overhang 16 in the antisense complex 10 and a single-stranded overhang 26 in the sense complex 22.

In one aspect, a terminal portion of the antisense 11 strand of the antisense complex 10 is single-stranded. In one aspect, a 3′ terminal portion of the antisense 11 strand of the antisense complex 10 is single-stranded. In one aspect, a 5′ terminal portion of the antisense 11 strand of the antisense complex 10 is single-stranded. In one aspect, a terminal portion of the sense 21 strand of the sense complex 20 is single-stranded. In one aspect, a 3′ terminal portion of the sense 21 strand of the sense complex 20 is single-stranded. In one aspect, a 5′ terminal portion of the sense 21 strand of the sense complex 20 is single-stranded. In one aspect, the single-stranded overhang is from about 1 to about 10 nucleotides in length, about 1 to about 5 nucleotides in length, about 1 to about 3 nucleotides in length, or about 1 to about 2 nucleotides in length. In one aspect, the single-stranded overhand is about 1, about 2, about 3, about 4, about 5, about 6, about 7, about 8, about 9, or about 10 nucleotides in length.

FIG. 2C is a schematic of a probe-probe binding complex 40 in which the “short” antisense binding portion 14 of the antisense probe 12 hybridizes to the “short” sense binding portion 24 of the sense probe 22. In this situation, when the antisense probe 10 has a “short” antisense binding portion 14 and the sense probe 20 has a “short” sense binding portion 24, there is a single-strand overhang 17 and 27 exposed in the probe-probe complex 40. In one aspect, the 5′ end of the antisense binding portion 14 of the antisense probe 12 in the probe-probe complex 40 is single-stranded. In one aspect, the 5′ end of the sense binding portion 24 of the sense probe 22 in the probe-probe complex 40 is single-stranded. In one aspect, the 5′ end of the antisense binding portion 14 of the antisense probe 12 and the 5′ end of the sense binding portion 24 of the sense probe 22 in the probe-probe complex 40 are each single-stranded. In one aspect, the 3′ end of the antisense binding portion 14 of the antisense probe 12 in the probe-probe complex 40 is single-stranded. In one aspect, the 3′ end of the sense binding portion 24 of the sense probe 22 in the probe-probe complex 40 is single-stranded. In one aspect, the 3′ end of the antisense binding portion 14 of the antisense probe 12 and the 3′ end of the sense binding portion 24 of the sense probe 22 in the probe-probe complex 40 are each single-stranded. In one aspect, the single-stranded overhang is from about 1 to about 10 nucleotides in length, about 1 to about 5 nucleotides in length, about 1 to about 3 nucleotides in length, or about 1 to about 2 nucleotides in length. In one aspect, the single-stranded overhand is about 1, about 2, about 3, about 4, about 5, about 6, about 7, about 8, about 9, or about 10 nucleotides in length.

FIG. 3A is a schematic of an antisense complex 10′ that includes an antisense strand 11 of an oligonucleotide duplex and an antisense probe 12′, wherein the antisense probe 12′ includes an oligonucleotide tag 13, a “full-length” antisense binding portion 14′ and a label 15. FIG. 3B is a schematic of a sense complex 20′ that includes a sense strand 21 from an oligonucleotide duplex and a sense probe 22′, wherein the sense probe 22′ includes an oligonucleotide tag 23, an “full-length” sense binding portion 24′ and a label 25. As used herein, the term “full-length” binding portion means that the binding portion of the antisense and sense probes are the same length (i.e., include the same number of nucleotide bases) as the antisense and sense strand of the oligonucleotide duplex, respectively, such that there is no single-stranded overhang in the antisense 10′ or sense complex 20′. FIG. 3C is a schematic of a probe-probe binding complex 40′ in which the “full-length” antisense binding portion 14′ of the antisense probe 12′ is hybridized to the “full-length” sense portion 24′ of the sense probe 22′, in which there is no single-stranded overhang.

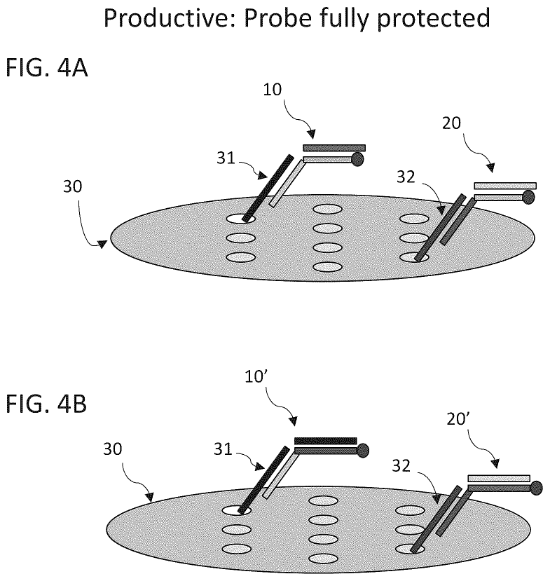

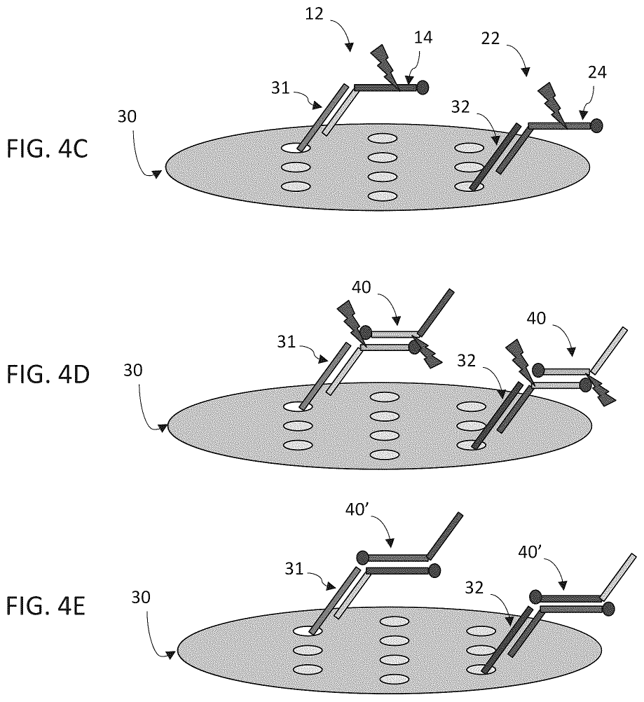

In one aspect, the support surface is contacted with the hybridization mixture under conditions in which the oligonucleotide tag of the sense or antisense probe hybridizes to a capture oligonucleotide immobilized on the support surface. In one aspect, the oligonucleotide tag of the antisense probe that is part of an antisense complex hybridizes to a capture oligonucleotide immobilized on the support surface. In one aspect, the oligonucleotide tag of the sense probe that is part of a sense complex hybridizes to a capture oligonucleotide immobilized on the support surface. In FIG. 4A, the antisense binding portion of the antisense probe is “short” such that the antisense strand portion of the antisense complex includes is a single-stranded overhang and the sense binding portion of the sense probe is “short” such that the sense strand portion of the sense complex includes a single-stranded overhang. In FIG. 4B, the antisense binding portion of the antisense probe is “full-length” such that the antisense strand portion of the antisense complex does not include a single-stranded overhang and the sense binding portion of the sense probe is “full-length” such that the sense strand portion of the sense complex does not include a single-stranded overhang. Situations in which an antisense or sense complex is hybridized to a support surface via the oligonucleotide tag of the antisense or sense probe, respectively, is referred to herein as “productive.”

In one aspect, the oligonucleotide tag of the antisense or sense probe is not part of an antisense or sense complex, respectively. Situations in which a hybridization complex that is not an antisense complex or a sense complex is hybridized to a support surface via the oligonucleotide tag of the antisense probe is referred to herein as “unproductive.” In one aspect, shown in FIG. 4C, the oligonucleotide tag of the antisense probe 12 or sense probe 22 binds only the antisense probe 12 or sense probe 22 to the support surface 30. In this situation, the antisense binding portion 14 and sense binding portion 24 of the antisense probe 12 or sense probe 22, respectively, remain single-stranded.

In one aspect, the oligonucleotide tag of the antisense or sense probe is part of a probe-probe complex and the oligonucleotide tag of the antisense probe 12 or sense probe 22 immobilizes a probe-probe complex 40′ onto the support surface 30. In one aspect, shown in FIG. 4D, the antisense-binding portion and sense-binding portion of the probes are “short” such that there is a single-strand overhang in the binding portions of the probe-probe complex 40. In one aspect, shown in FIG. 4E, the antisense-binding portion and sense-binding portion of the probes are “full-length” such that there is no single-strand overhang in the binding portions of the probe-probe complex 40′.

In one aspect, the sample includes a plurality of oligonucleotide duplexes and the composition includes a plurality of sets of probes, wherein each set of probes hybridizes with a unique sense or antisense strand of a unique oligonucleotide duplex.

In one aspect, the method includes step-down hybridization conditions, in which the probes hybridize to their respective sense or antisense strands during incremental reductions in annealing temperature. In one aspect, the hybridization conditions include a denaturing step in which the sample is incubated a first temperature to denature the sense and antisense strands of the oligonucleotide duplex. In one aspect, the hybridization conditions include an annealing step in which the probes are incubated with the denatured sense and antisense strands of the oligonucleotide duplex at a second temperature to allow the sense and antisense probes to hybridize to the sense and antisense strands. In one aspect, the method includes incubating the sense and antisense complexes at a hold temperature of about 2° C. to about 8° C.

In one aspect, the denaturing step includes incubating the sample at a first temperature from about 60° C. to about 95° C. to denature the sense and antisense strands of the oligonucleotide duplex. In one aspect, the denaturing step includes incubating the sample at a first temperature of at least about 60° C., about 65° C., about 70° C., about 75° C., or about 80° C. and up to about 85° C., about 90° C., about 95° C., or about 100° C. In one aspect, the denaturing step includes incubating the sample at a first temperature of about 60° C., about 65° C., about 70° C., about 75° C., about 80° C., about 85° C., about 90° C., about 95° C., or about 100° C. In one aspect, the hybridization conditions include incubating the sample at a first temperature of about 80° C. to about 95° C. to denature the sense and antisense strands of the oligonucleotide duplex. In one aspect, the hybridization conditions include incubating the sample at a first temperature of about 90° C. to about 95° C. to denature the sense and antisense strands of the oligonucleotide duplex. In one aspect, the hybridization conditions include incubating the sample at a first temperature of about 95° C. to denature the sense and antisense strands of the oligonucleotide duplex. In one aspect, the sample is incubated for about 1 minute to about 15 minutes. In one aspect, the sample is incubated for at least about 30 seconds, about 1 minute, about 2 minutes, about 3 minutes, about 4 minute or about 5 minutes and up to about 10 minutes or about 15 minutes. In one aspect, the sample is incubated for about 30 seconds, about 1 minute, about 2 minutes, about 3 minutes, about 4 minutes, about 5 minutes, about 10 minutes or about 15 minutes. In one aspect, the sample is incubated from about 1 minute to about 5 minutes. In one aspect, the sample is incubated from about 1 minute to about 2 minutes. In one aspect, the sample is incubated for about 2 minutes. In one aspect, the denaturing step includes incubating the probes with the sample a first temperature from about 60° C. to about 95° C. for about 1 minute to about 15 minutes to denature the sense and antisense strands of the oligonucleotide duplex. In one aspect, the hybridization conditions include incubating the probes with the sample a first temperature of about 80° C. to about 95° C. for about 1 minute to about 5 minutes to denature the sense and antisense strands of the oligonucleotide duplex. In one aspect, the hybridization conditions include incubating the probes with the sample a first temperature of about 95° C. for about 2 minutes to denature the sense and antisense strands of the oligonucleotide duplex.

In one aspect, the annealing step includes incubating the probes with the sample at a second temperature from about 10° C. to about 65° C. to allow the sense and antisense probes to hybridize to the sense and antisense strands. In one aspect, the annealing step includes incubating the probes with the sample at a second temperature of at least about 10° C., about 15° C., about 20° C., about 25° C., about 30° C., about 35° C., or about 40° C. and up to about 45° C., about 50° C., about 55° C., about 60° C., or about 65° C. to allow the sense and antisense probes to hybridize to the sense and antisense strands. In one aspect, the annealing step includes incubating the probes with the sample at a second temperature of about 10° C., about 15° C., about 20° C., about 25° C., about 30° C., about 35° C., about 40° C., about 45° C., about 50° C., about 55° C., about 60° C., or about 65° C. to allow the sense and antisense probes to hybridize to the sense and antisense strands. In one aspect, the hybridization conditions include incubating the probes with the sample at a second temperature from about 40° C. and about 65° C. to allow the sense and antisense probes to hybridize to the sense and antisense strands. In one aspect, the hybridization conditions include incubating the probes with the sample at a second temperature of 60° C. to about 65° C. to allow the sense and antisense probes to hybridize to the sense and antisense strands. In one aspect, the hybridization conditions include incubating the probes with the sample at a second temperature of about 650 to allow the sense and antisense probes to hybridize to the sense and antisense strands. In one aspect, the annealing step includes incubating the probes with the sample at a second temperature for about 30 seconds to about 5 minutes to allow the sense and antisense probes to hybridize to the sense and antisense strands. In one aspect, the annealing step includes incubating the probes with the sample at a second temperature for at least about 30 seconds, about 60 seconds, about 90 seconds and up to about 2 minutes, about 3 minutes, about 4 minutes or about 5 minutes to allow the sense and antisense probes to hybridize to the sense and antisense strands. In one aspect, the hybridization conditions include incubating the probes with the sample at a second temperature for about 1 minute to about 2 minutes to allow the sense and antisense probes to hybridize to the sense and antisense strands. In one aspect, the hybridization conditions include incubating the probes with the sample at a second temperature to allow the sense and antisense probes to hybridize to the sense and antisense strands. In one aspect, the annealing step includes incubating the probes with the sample at a second temperature from about 10° C. to about 65° C. for about 30 seconds to about 5 minutes to allow the sense and antisense probes to hybridize to the sense and antisense strands. In one aspect, the hybridization conditions include incubating the probes with the sample at a second temperature from about 40° C. and about 65° C. for about 1 minute to about 2 minutes to allow the sense and antisense probes to hybridize to the sense and antisense strands. In one aspect, the hybridization conditions include incubating the probes with the sample at a second temperature of about 65° C. for about 1 min to allow the sense and antisense probes to hybridize to the sense and antisense strands. In one aspect, the hybridization conditions include incubating the probes with the sample at a hold temperature of about 2° C. to about 8° C. In one aspect, the hybridization conditions include incubating the probes with the sample at a hold temperature of about 4° C.

In one aspect, the temperature transition rate between the annealing step and the hold is from about 0.05° C./s to about 0.5° C./s. In one aspect, the temperature transition rate between the annealing step and the hold is about 0.1° C./s.

In one aspect, the probes are incubated with the sample in a buffer that includes diluent 54 or N-PLEX hybridization Buffer 1 or 2.

In one aspect, the hybridization mixture containing the hybridization complexes is contacted with a single-strand specific nuclease. In one aspect, the hybridization mixture containing the hybridization complexes is contacted with a lysis buffer. In one aspect, the support surface is first contacted with the hybridization mixture under conditions in which the first and second oligonucleotide tags of the sense and antisense probes hybridize to the first and second capture oligonucleotides on the support surface to immobilize the hybridization complexes on the support surface; and then the support surface is contacted with a single-strand specific nuclease, a lysis buffer, or both. In one aspect, the hybridization mixture containing the hybridization complexes is contacted with a single-strand specific nuclease, a lysis buffer, or both, to form a reaction mixture; and then the support surface is contacted with the reaction mixture under conditions in which the oligonucleotide tags of the sense and antisense probes hybridize to the capture oligonucleotides immobilized on the support surface. In one aspect, the support surface is contacted with a single-strand specific nuclease at about 25° C. to about 40° C. In one aspect, the support surface is contacted with a single-strand specific nuclease at less than 37° C. In one aspect, the support surface is contacted with a single-strand specific nuclease at about 27° C. to about 37° C. In one aspect, the support surface is contacted with a single-strand specific nuclease at about 30° C. or 37° C. In one aspect, the support surface is contacted with a single-strand specific nuclease for about 1 to about 90 minutes, for about 30 to 90 minutes, for about 30 minutes or for about 60 minutes. In one aspect, the single-strand specific nuclease includes a single-strand specific DNase. In one aspect, the single-strand specific DNase is S1 nuclease, P1 nuclease or Mung Bean nuclease. In one aspect, the single-strand specific nuclease includes a single-strand specific RNase. In one aspect, the single-strand specific RNase is RNase A, RNase H, RNase I, RNase III, RNase L, RNase P, RNase PhyM, RNase T1, RNase T2, RNase U2, RNase V, PNPase, RNase PH, RNase R, RNase D, RNase T, RNaseONE, oligoribonuclease, exoribonuclease I, or exoribonuclease II.

In one aspect, the lysis buffer comprises sodium dodecyl sulphate (SDS), lithium dodecyl sulphate (SDS), Triton X (100, 114), NP-40, Tween (20, 80), Cetyltrimethylammonium bromide (CTAB), CHAPS, CHAPSO, Proteinase K, or any combination thereof. It is to be understood that lysis buffers are known to one of ordinary skill in the art. In one aspect, the lysis buffer includes a diluent, lithium dodecyl sulfate (LDS), and/or Proteinase K. In one aspect, the lysis buffer includes a diluent, 2% lithium dodecyl sulfate (LDS), and 0.4 mg/mL Proteinase K.

In one aspect, shown in FIG. 4C, the single-stranded nuclease cleaves unbound single-stranded probe in the hybridization complexes, detaching the label from the probe. Advantageously, this removes the label from the probe and reduces background caused by immobilization of the unbound probes to the support surface.

In one aspect, shown in FIG. 4D, the single-stranded nuclease cleaves the single-stranded overhang the probe-probe complex formed using “short” probes, removing label from the probe-probe complex and thereby reducing background levels.

In one aspect, shown in FIG. 4E, the probe-probe complex is formed from FL probes such that there is no single stranded overhang. In this situation, the labels remain immobilized on the support surface, resulting in high background levels.

In one aspect, one or more of the method steps of contacting the sample with the composition that includes the set of probes, incubating the probes with the sample to form hybridization complexes that include a sense complex and an antisense complex, contacting a support surface with the hybridization mixture that includes the hybridization complexes and contacting the support surface with a single-strand specific nuclease are performed concurrently. In one aspect, the method steps of contacting the sample with the composition that includes the set of probes, incubating the probes with the sample to form hybridization complexes that includes a sense complex and an antisense complex, contacting a support surface with the hybridization mixture that includes the hybridization complexes and contacting the hybridization complexes with a single-strand specific nuclease are all performed concurrently.

In one aspect, one or more of the method steps of contacting the sample with the composition that includes the set of probes, incubating the probes with the sample to form a hybridization mixture that includes hybridization complexes that include a sense complex and an antisense complex, contacting a support surface with the hybridization mixture and contacting the hybridization complexes with a single-strand specific nuclease are performed sequentially. In one aspect, the method steps of contacting the sample with the composition that includes the set of probes, incubating the probes with the sample to form a hybridization mixture that includes hybridization complexes that includes a sense complex and an antisense complex, contacting a support surface with the hybridization mixture and contacting the hybridization complexes with a single-strand specific nuclease are each performed sequentially.

In one aspect, the support surface is incubated with the hybridization complexes for about 15 minutes to about 12 hours. In one aspect, the support surface is incubated with the hybridization complexes for at least about 15 minutes, about 30 minutes, about 1 hour, about 2 hours, about 3 hours, about 4 hours, about 5 hours, or about 6 hours and up to about 6 hours or about 12 hours. In one aspect, the support surface is incubated with the hybridization complexes for about 30 minutes to about 3 hours. In one aspect, the support surface is incubated with the hybridization complexes for about 1 hour to about 2 hours.

In one aspect, the support surface is incubated with the hybridization complexes at a temperature of about 20° C. to about 40° C. In one aspect, the support surface is incubated with the hybridization complexes at a temperature of at least about 20° C., about 25° C., or about 30° C., and up to about 25° C., or about 40° C. In one aspect, the support surface is incubated with the hybridization complexes at a temperature of about 20° C. to about 40° C. In one aspect, the support surface is incubated with the hybridization complexes at a temperature of about 30° C. to about 40° C. In one aspect, the support surface is incubated with the hybridization complexes at a temperature of less than about 37° C. In one aspect, the support surface is incubated with the hybridization complexes at a temperature of about 27° C. to about 37° C. In one aspect, the support surface is incubated with the hybridization complexes at a temperature of about 35° C. to about 40° C. In one aspect, the support surface is incubated with the hybridization complexes at a temperature of about 30° C., about 31° C., about 32° C., about 33° C., about 34° C., about 35° C., about 36° C., about 37° C., about 38° C., about 39° C., or about 40° C. In one aspect, the support surface is incubated with the sense and antisense complexes at a temperature of about 30° C. or 37° C.

In one aspect, the support surface is incubated with the hybridization complexes while shaking. while shaking at about 700 rpm to about 900 rpm. In one aspect, the support surface is incubated with the hybridization complexes while shaking. while shaking at from about 700 rpm, 705 rpm, 710 rpm, 725 rpm, 750 rpm and up to about 800 rpm, about 850 rpm or about 900 rpm. In one aspect, the support surface is incubated with the hybridization complexes while shaking. at about 705 rpm.

In one aspect, the support surface is incubated with the hybridization complexes for about 15 minutes to about 12 hours at a temperature of about 20° C. to about 40° C. In one aspect, the support surface is incubated with the hybridization complexes for about 1 hour to about 2 hours at a temperature of about 20° C. to about 40° C. In one aspect, the support surface is incubated with the sense and antisense complexes while shaking. In one aspect, the support surface is incubated with the sense and antisense complexes for about 1 hour at a temperature of about 37° C. or 30° C., while shaking at about 705 rpm.

In one aspect, the sample includes a plurality of oligonucleotide duplexes and the probe composition includes a plurality of sets of probes, in which each set of probes hybridizes with a unique sense or antisense strand of a unique oligonucleotide duplex. In one aspect, the probe composition includes about 20 pM to about 10 nM sense probe. In one aspect, the probe composition includes from about 20 pM, about 50 pM, about 100 pM, about 150 pM, about 200 pM, or about 250 pM and up to about 0.5 nM, about 1 nM, about 5 nM, or about 10 nM sense probe. In one aspect, the probe composition includes about 100 pM to about 500 pM sense probe. In one aspect, the probe composition includes about 20 pM to about 200 pM sense probe. In one aspect, the probe composition includes about 20 pM to about 100 pM sense probe. In one aspect, the probe composition includes from about 20 pM, about 50 pM, about 100 pM, about 150 pM, about 200 pM or about 250 pM, and up to about 0.5 nM, about 1 nM, about 5 nM, or about 10 nM antisense probe. In one aspect, the probe composition includes about 100 pM to about 500 pM antisense probe. In one aspect, the probe composition includes about 20 pM to about 200 pM antisense probe.

In one aspect, the method includes detecting or quantifying the sense and antisense strands of the oligonucleotide duplex based on the presence the label immobilized on the support surface. In one aspect, the method has a lower limit of quantitation (LLOQ) of less than about 20 pM. In one aspect, the LLOQ is less than about 15 pM for detecting both AS and SS strands. In one aspect, the LLOQ is less than about 10 pM for detecting both AS and SS strands. In one aspect, the LLOQ is less than about 1 pM for detecting both AS and SS strands. In one aspect, the LLOQ is greater than about 0.01 pM for detecting both AS and SS strands. In one aspect, the lower limits of detection (LLODs) are less than about 5 pM for both SS and AS strands. In one aspect, the LLODs are less than about 1 pM and greater than about 0.01 pM for both SS and AS strands. In one aspect, the LLODs are 0.8 pM for both SS and AS strands.

### C. Oligonucleotide Duplex

In one aspect, the method described herein is used to detect an oligonucleotide duplex in a sample. In one aspect, the method described herein is used to detect a first and second strand of an oligonucleotide duplex. In one aspect, the oligonucleotide duplex is a double-stranded oligonucleotide therapeutic. In one aspect, the oligonucleotide therapeutic includes a sense and an antisense strand.

In one aspect, both strands of the oligonucleotide duplex include DNA. In one aspect, the oligonucleotide duplex is a DNA/DNA duplex. In one aspect, both strands of the oligonucleotide duplex include RNA. In one aspect the oligonucleotide duplex is an RNA/RNA duplex. In one aspect, one strand of the oligonucleotide duplex includes DNA and one strand of the oligonucleotide duplex includes RNA. In one aspect, the oligonucleotide duplex is a DNA/RNA heteroduplex. In one aspect, the sense strand of the oligonucleotide duplex includes DNA. In one aspect, the sense strand of the oligonucleotide duplex includes RNA. In one aspect, the antisense strand of the oligonucleotide duplex includes DNA. In one aspect, the antisense strand of the oligonucleotide duplex includes RNA.

In one aspect, one or both strands of the oligonucleotide duplex include one or more modified nucleotides. In one aspect, one of the strands of the oligonucleotide duplex includes DNA and one or more modified nucleotides. In one aspect, one of the strands of the oligonucleotide duplex includes RNA and one or more modified nucleotides. In one aspect, the antisense strand of the oligonucleotide duplex includes DNA and one or more modified nucleotides. In one aspect, the antisense strand of the oligonucleotide duplex includes RNA and one or more modified nucleotides. In one aspect, the sense strand of the oligonucleotide duplex includes DNA and one or more modified nucleotides. In one aspect, the sense strand of the oligonucleotide duplex includes RNA and one or more modified nucleotides. In one aspect, the modified nucleotide includes a modification at a nucleobase, a sugar or an internucleotidic linkage. In one aspect, the sense strand, antisense strand or both the sense and antisense strands of the oligonucleotide duplex individually include one or more modified nucleic acids. In one aspect, the sense strand, the antisense strand or both the sense and antisense strands of the oligonucleotide duplex individually include a 5′- or 3′-bioconjugate. In one aspect, the bioconjugate includes polyethylene glycol (PEG), N-acetylgalactosamine (GalNAc), a cell penetrating peptide (CPP), α-tocopherol, an aptamer, an antibody, cholesterol, squalene, a fatty acid, or a nucleolipid.

In one aspect, each strand of the oligonucleotide duplex independently includes from about 5 to about 100 nucleotides. In one aspect, each strand of the oligonucleotide duplex independently includes from about 5, about 6, about 7, about 8, about 9, about 10, about 11, about 12, about 13, about 14, about 15, about 20, about 25 or about 30 nucleotides and up to about 30, about 35, about 40, about 45, about 50 or about 100 nucleotides in length. In one aspect, each strand of the oligonucleotide duplex includes from about 8 to about 50 nucleotides, about 10 to about 40 nucleotides, about 10 to about 30 nucleotides, about 12 to about 30 nucleotides, about 16 to about 30, or about 18 to about 30 nucleotides.

### D. Oligonucleotide Probes

In one aspect, a sense probe is provided that is capable of hybridizing to a sense strand of an oligonucleotide duplex. In one aspect, an antisense probe is provided that is capable of hybridizing to an antisense strand of an oligonucleotide duplex. In one aspect, an antisense probe is provided that includes an oligonucleotide tag, an antisense-binding portion and a label. In one aspect, a sense probe is provided that includes an oligonucleotide tag, a sense-binding portion and a label.

### 1. Oligonucleotide Tag

In one aspect, the sense and antisense probes include an oligonucleotide tag having a nucleic acid sequence that hybridizes to an oligonucleotide sequence of a capture oligonucleotide. In one aspect, the oligonucleotide tag includes a single-stranded oligonucleotide that is complementary to at least a portion of the nucleotide sequence of a single stranded capture oligonucleotide. In one aspect, the oligonucleotide tag hybridizes to its corresponding capture oligonucleotide.

In one aspect, the oligonucleotide tag does not cross-react with or hybridize to capture oligonucleotides that are not its corresponding capture oligonucleotide. In one aspect, the oligonucleotide tag does not cross-react with or hybridize to capture oligonucleotides that are not its corresponding capture oligonucleotide under stringent hybridization conditions. In one aspect, the oligonucleotide tag does not hybridize to the sense or antisense strand of the oligonucleotide duplex. In one aspect, the oligonucleotide tag of the sense probe does not hybridize to the antisense binding portion of the antisense probe, or vice versa. In one aspect, the oligonucleotide tag of the sense probe does not hybridize to the antisense binding portion of the antisense probe under physiologically relevant or stringent conditions. In one aspect, the oligonucleotide tag of the antisense probe does not hybridize to the sense binding portion of the sense probe under physiologically relevant or stringent conditions. In one aspect, the oligonucleotide tag does not hybridize to the sense or antisense strand of the oligonucleotide duplex under physiologically relevant or stringent conditions.

In one aspect, the oligonucleotide tag and its corresponding capture oligonucleotide have 100% “sequence complementarity” based on the Watson-Crick model. In one aspect, the oligonucleotide tag and the capture oligonucleotide have sequences that have at least about 90%, about 95%, about 96%, about 97%, about 98% or about 99% sequence complementarity based on the Watson-Crick model.

In one aspect, the oligonucleotide tag is recombinantly produced. In one aspect, the oligonucleotide tag is chemically synthesized. In one aspect, the oligonucleotide tags are not naturally occurring sequences. In one aspect, the oligonucleotide tag includes a single stranded DNA sequence. In one aspect, the oligonucleotide tag includes a single stranded RNA sequence. In one aspect, the oligonucleotide tag of the sense probe includes RNA. In one aspect, the oligonucleotide tag of the antisense probe includes RNA. In one aspect, the oligonucleotide tag of the sense probe includes DNA. In one aspect, the oligonucleotide tag of the antisense probe includes DNA.

In one aspect, the oligonucleotide tag of the antisense probe includes one or more modified nucleic acids. In one aspect, the oligonucleotide tag of the antisense probe includes one or more modified nucleotides selected from phosphodiester (PO); phosphorothioate (PS); 2′O-methyl (2′OMe); 2′O-methoxyethyl (MOE); peptide nucleic acid (PNA); phosphoroamidate morpholino (PMO); locked nucleic acid (LNA); 2′-deoxy-2′-fluoro (2′-F); or a combination thereof. In one aspect, the oligonucleotide tag of the antisense probe includes a locked nucleic acid (LNA).

In one aspect, the oligonucleotide tag of the sense probe includes one or more modified nucleic acids. In one aspect, the oligonucleotide tag of the sense probe includes one or more modified nucleotides selected from phosphodiester (PO); phosphorothioate (PS); 2′O-methyl (2′OMe); 2′O-methoxyethyl (MOE); peptide nucleic acid (PNA); phosphoroamidate morpholino (PMO); locked nucleic acid (LNA); 2′-deoxy-2-fluoro (2′-F); or a combination thereof. In one aspect, the oligonucleotide tag of the sense probe includes a locked nucleic acid (LNA).

In one aspect, the oligonucleotide tag includes one or more modified nucleotides.

In one aspect, the oligonucleotide tag is attached to the 5′-end of the antisense probe. In one aspect, the oligonucleotide tag is attached to the 3′-end of the antisense probe. In one aspect, the oligonucleotide tag is attached to the 5′-end of the sense probe. In another aspect, the oligonucleotide tag is attached to the 3′-end of the sense probe. In one aspect, the oligonucleotide tag is not complementary to and does not hybridize with the sense or antisense strand of the oligonucleotide duplex.

In one aspect, the oligonucleotide tag has a nucleotide sequence that is at least about 15, about 16, about 17, about 18, about 19, about 20, about 21, about 22, about 23, about 24 or about 25 and up to about 25, about 26, about 27, about 28, about 29, about 30, about 31, about 32, about 33, about 34, about 35, about 36, about 37, about 38, about 39 or about 40, or from about 15 and about 40, or about 20 and about 30 nucleotides in length. In one aspect, the oligonucleotide tag includes a nucleotide sequence that is at least about 1, about 2, about 3, about 4, about 5, about 6, about 7, about 8, about 9, or about 10 and up to about 11, about 12, about 13, about 14, about 15, about 16, about 17, about 18, about 19, or about 20, or from about 1 to about 20, from about 10 to about 15, or from about 12 to about 13 nucleotides shorter than the complementary capture oligonucleotide sequence. In one aspect, the tag has a nucleotide sequence that is at least about 24, about 30 or about 36 nucleotides in length. In one aspect, the oligonucleotide tag has a length that is the same as the length of the corresponding capture oligonucleotide. In one aspect, the oligonucleotide tag has a length that is the shorter than the length of the corresponding capture oligonucleotide.

### 2. Binding Portion

In one aspect, the antisense probe includes an antisense binding portion. In one aspect, the antisense binding portion of the antisense probe has a nucleic acid sequence that is complementary to a nucleic acid sequence of an antisense strand of an oligonucleotide duplex. In one aspect, the antisense binding portion of the antisense probe is capable of hybridizing to an antisense strand of an oligonucleotide duplex. In one aspect, the antisense binding portion and the antisense strand of the oligonucleotide duplex have 100% “sequence complementarity” based on the Watson-Crick model. In one aspect, the antisense binding portion and the antisense strand of the oligonucleotide duplex have sequences that have at least about 90%, about 95%, about 96%, about 97%, about 98% or about 99% sequence complementarity based on the Watson-Crick model.

In one aspect, the sense probe includes a sense binding portion. In one aspect, the sense binding portion of the sense probe has a nucleic acid sequence that is complementary to a nucleic acid sequence of a sense strand of an oligonucleotide duplex. In one aspect, the sense binding portion of the sense probe is capable of hybridizing to a sense strand of an oligonucleotide duplex. In one aspect, the sense binding portion and the sense strand of the oligonucleotide duplex have 100% “sequence complementarity” based on the Watson-Crick model. In one aspect, the sense binding portion and the sense strand of the oligonucleotide duplex have sequences that have at least about 90%, about 95%, about 96%, about 97%, about 98% or about 99% sequence complementarity based on the Watson-Crick model.

In one aspect, the antisense binding length of the antisense probe is shorter than the antisense strand length of the antisense strand by at least 1 nucleotide. In one aspect, the antisense binding length of the antisense probe is shorter than the antisense strand length of the antisense strand by at least 2 nucleotides. In one aspect, the antisense binding portion has a length that is from about 1 to about 10 nucleotides shorter than the antisense strand of the oligonucleotide duplex. In one aspect, the antisense binding portion has a length that is from about 1 to about 5 nucleotides shorter than the antisense strand of the oligonucleotide duplex. In one aspect, the antisense binding portion has a length of about 10 to about 25 nucleotides, or about 10 to about 20 nucleotides, or about 10 to about 16 nucleotides in length. In one aspect, the antisense binding portion of the antisense probe has a length this is from about 50% to about 99% of the length of the antisense strand of the oligonucleotide duplex. In one aspect, the antisense binding portion of the antisense probe has a length this is from about 75% to about 95% of the length of the antisense strand of the oligonucleotide duplex. In one aspect, the antisense binding portion has a length of about 80%, of about 85%, of about 90% or of about 100% of the length of the antisense strand of the oligonucleotide duplex. In one aspect, the antisense binding portion has a length of about 80% of the length of the antisense strand of the oligonucleotide duplex.

In one aspect, the sense binding length of the sense probe is shorter than the sense strand length of the sense strand by at least 1 nucleotide. In one aspect, the sense binding length of the sense probe is shorter than the sense strand length of the sense strand by at least 2 nucleotides. In one aspect, the sense binding portion has a length that is from about 1 to about 10 nucleotides shorter than the sense strand of the oligonucleotide duplex. In one aspect, the sense binding portion has a length that is from about 1 to about 5 nucleotides shorter than the sense strand of the oligonucleotide duplex. In one aspect, the sense binding portion has a length of about 10 to about 25 nucleotides, or about 10 to about 20 nucleotides, or about 10 to about 16 nucleotides in length. In one aspect, the sense binding portion of the sense probe has a length this is from about 50% to about 99% of the length of the sense strand of the oligonucleotide duplex. In one aspect, the sense binding portion of the sense probe has a length this is from about 75% to about 95% of the length of the sense strand of the oligonucleotide duplex. In one aspect, the sense binding portion has a length of about 80%, of about 85%, of about 90% or of about 100% of the length of the sense strand of the oligonucleotide duplex. In one aspect, the sense binding portion has a length of about 80% of the length of the sense strand of the oligonucleotide duplex.

In one aspect, the sense binding portion of the sense probe has a 5′ end that aligns with a 3′ end of the sense strand of the oligonucleotide duplex. In one aspect, the sense binding portion of the sense probe has a 3′ end that aligns with a 5′ end of the sense strand of the oligonucleotide duplex. In one aspect, the antisense binding portion of the antisense probe has a 5′ end that aligns with a 3′ end of the antisense strand of the oligonucleotide duplex. In one aspect, the antisense binding portion of the antisense probe has a 3′ end that aligns with a 5′ end of the antisense strand of the oligonucleotide duplex.

In one aspect, the antisense binding portion of the antisense probe includes DNA. In one aspect, the antisense binding portion of the antisense probe includes RNA. In one aspect, the sense binding portion of the sense probe includes DNA. In one aspect, the sense binding portion of the sense probe includes RNA.

In one aspect, the antisense binding portion and the oligonucleotide tag of the antisense probe include DNA. In one aspect, the antisense binding portion and the oligonucleotide tag of the antisense probe include RNA. In one aspect, the antisense binding portion of the antisense probe includes DNA and the oligonucleotide tag of the antisense probe includes RNA. In one aspect, the antisense binding portion of the antisense probe includes RNA and the oligonucleotide tag of the antisense probe includes DNA.

In one aspect, the sense binding portion and the oligonucleotide tag of the sense probe include DNA. In one aspect, the sense binding portion and the oligonucleotide tag of the sense probe include RNA. In one aspect, the sense binding portion of the sense probe includes DNA and the oligonucleotide tag of the sense probe includes RNA. In one aspect, the sense binding portion of the sense probe includes RNA and the oligonucleotide tag of the sense probe includes DNA.

In one aspect, the antisense probe is a chimeric probe that includes an antisense binding portion that includes DNA and an oligonucleotide tag that includes RNA, wherein the antisense binding portion the antisense probe has an antisense binding length that is shorter than the antisense strand length of the antisense strand of the oligonucleotide duplex by at least 1 nucleotide. In one aspect, the antisense probe is a chimeric probe that includes an antisense binding portion that includes RNA and an oligonucleotide tag that includes DNA, wherein the antisense binding portion the antisense probe has an antisense binding length that is shorter than the antisense strand length of the antisense strand of the oligonucleotide duplex by at least 1 nucleotide. In one aspect, the sense probe is a chimeric probe that includes a sense binding portion that includes DNA and an oligonucleotide tag that includes RNA, wherein the sense binding portion the sense probe has a sense binding length that is shorter than the sense strand length of the sense strand of the oligonucleotide duplex by at least 1 nucleotide. In one aspect, the sense probe is a chimeric probe that includes a sense binding portion that includes RNA and an oligonucleotide tag that includes DNA, wherein the sense binding portion the sense probe has a sense binding length that is shorter than the sense strand length of the sense strand of the oligonucleotide duplex by at least 1 nucleotide.

In one aspect, the binding portion of the antisense probe includes one or more modified nucleic acids. In one aspect, the binding portion of the antisense probe includes one or more modified nucleotides selected from phosphodiester (PO); phosphorothioate (PS); 2′O-methyl (2′OMe); 2′O-methoxyethyl (MOE); peptide nucleic acid (PNA); phosphoroamidate morpholino (PMO); locked nucleic acid (LNA); 2′-deoxy-2′-fluoro (2′-F); or a combination thereof. In one aspect, the binding portion of the antisense probe includes a locked nucleic acid (LNA).

In one aspect, the binding portion of the sense probe includes one or more modified nucleic acids. In one aspect, the binding portion of the sense probe includes one or more modified nucleotides selected from phosphodiester (PO); phosphorothioate (PS); 2′O-methyl (2′OMe); 2′O-methoxyethyl (MOE); peptide nucleic acid (PNA); phosphoroamidate morpholino (PMO); locked nucleic acid (LNA); 2′-deoxy-2′-fluoro (2′-F); or a combination thereof. In one aspect, the binding portion of the sense probe includes a locked nucleic acid (LNA).

### 3. Label

In one aspect, the probe includes a label. In one aspect, the label is attached directly to the probe. In another aspect, the label is attached to the probe through a linker. In one aspect, the label is attached to the 5′ end of the probe. In one aspect, the label is attached to the 3′ end of the probe. In one aspect, the label is a primary label. In one aspect, the primary label has a detectable physical property. Examples or primary labels include, but are not limited to, radioisotopes, enzymes, substrates, fluorescent molecules, chemiluminescent moieties, electrochemiluminescent (ECL) moieties, magnetic particles, and bioluminescent moieties. In one aspect, the primary label is an electrochemiluminescence (ECL) label. In one aspect, the ECL label is an organometallic complex that includes a transition metal, for example, ruthenium. In one aspect, the primary label is a MSD SULFO-TAG label (Meso Scale Diagnostics, LLC, Rockville, MD, U.S.A.).

In one aspect, the label is a compound that is a member of a binding pair, in which a first member of the binding pair (which can be referred to as a “primary binding reagent”) is attached to a substrate, for example, an oligonucleotide, and the other member of the binding pair (which can be referred to as a “secondary binding reagent”) has a detectable physical property or is attached to a moiety that has a detectable physical property. Non-limiting examples of binding pairs include biotin and streptavidin, or avidin; complementary oligonucleotides; hapten and hapten binding partner; and antibody-antigen binding pairs. In one aspect, the label is a primary binding agent that includes biotin. In one aspect, the secondary binding reagent includes streptavidin. In one aspect, the secondary binding reagent includes a MSD SULFO-TAG label (Meso Scale Diagnostics, LLC, Rockville, MD, U.S.A.).

### E. Samples

In one aspect, the oligonucleotide duplex is in a sample. In one aspect, the sample is a biological sample obtained or derived from a source of interest. In one aspect, a sample is an organism or is obtained from an organism. In one aspect, a sample is a plant or is obtained from a plant. In one aspect, the sample is an animal or is obtained from an animal. In one aspect, the sample is obtained from a mammal. In one aspect, the sample is obtained from a human. In one aspect, the source of interest includes a bioreactor. In one aspect, the sample is a manufacturing process sample. In one aspect, the sample is an environmental sample. In one aspect, the environmental sample includes a water sample, including, for example, an aquifer sample, a ground water sample, or a waste water sample. In one aspect, the environmental sample includes a soil sample. In one aspect, the environmental sample includes a soil microorganism sample. In one aspect, the sample includes cell-free DNA.

In one aspect, the sample includes a biological sample. In one aspect, the sample includes an untreated biological sample. In one aspect, the sample includes a pretreated biological sample. In one aspect, the sample is pretreated, for example, to remove one or more components or to add one or more agents. In one aspect, the sample includes a purified sample. Methods of purifying oligonucleotides from a biological sample are known and include, for example, precipitation, centrifugation, and column chromatography. In one aspect, column chromatography includes high performance liquid chromatography (HPLC), for example, reverse phase high performance liquid chromatography (RP-HPLC), anion exchange high pressure liquid chromatography (AEX HPLC) or polyacrylamide gel electrophoresis (PAGE). In one aspect, the sample is an extracted sample. In one aspect, the sample is filtered, for example, using a semi-permeable membrane. In one aspect, the sample includes oligonucleotides extracted from a sample or obtained by subjecting a sample to techniques such as amplification or reverse transcription of mRNA.

In one aspect, the sample includes one or more target oligonucleotide sequences. In one aspect, the sample includes one or more amplified target oligonucleotide sequences. In one aspect, the sample includes one or more amplified target oligonucleotide sequence obtained by methods including, but not limited to, polymerase chain reaction (PCR) or rolling circle amplification (RCA).

In one aspect, the biological sample includes biological tissue or fluid, including, for example, body fluids, secretions, excretions, cells, tissues or organs or homogenates thereof. In one aspect, the biological fluid includes plasma, serum, whole blood, lymph, urine, feces, breast milk, sputum, saliva, ascites, cerebrospinal fluid, peritoneal fluid, pleural fluid and amniotic fluid. In one aspect, the biological tissue includes tissue or tissue homogenates, including, but not limited to, organs or organ homogenates, such as brain, liver, spleen, heart, lung, and kidney or other tissues, for example, muscle, skin, or bone marrow. In one aspect, the biological sample includes tissue or fine needle biopsy samples, cell-containing body fluids, free floating nucleic acids, gynecological fluids, skin swabs, vaginal swabs, oral swabs, nasal swabs, washings or lavages such as a ductal lavages or bronchoalveolar lavages, aspirates, scrapings; surgical specimens.

In one aspect, the biological sample includes a sample isolated from a portion of the human body. In one aspect, biological sample includes a sample isolated from a nasal passage, oral cavity, skin, ear, mucus membrane, gastrointestinal tract, urogenital tract, respiratory tract, eye or a combination thereof. In one aspect, the biological sample includes a sample obtained from a portion of the human body using methods known in the art, including, but not limited to swabbing, puncture sampling, and serum sampling.

In one aspect, the sample includes a naturally occurring RNase. For samples that include a naturally occurring RNase, it may be desirable to contact the sample with an RNase inhibitor before the sample is contacted with the sense and antisense probes.

### F. Capture Oligonucleotide

In one aspect, the method or kit includes one or more capture oligonucleotides that are or can be immobilized in discrete binding domains on a support surface. In one aspect, the capture oligonucleotides are not naturally occurring sequences. In another aspect, the capture oligonucleotides are recombinantly produced. In one aspect, the capture oligonucleotides are chemically synthesized.

In one aspect, the capture oligonucleotides are single stranded capture oligonucleotides having nucleotide sequences that are complementary to a nucleotide sequence of a single stranded oligonucleotide tag. In one aspect, the oligonucleotide tag is attached to a sense or an antisense probe.

In one aspect, the method or kit includes a unique capture oligonucleotide the sense and antisense strand of an oligonucleotide duplex of interest. In one aspect, the capture oligonucleotides are immobilized on a support surface. In one aspect, the capture oligonucleotides are immobilized in an array. In one aspect, the array includes two or more capture oligonucleotides. In one aspect, the array includes from about 2 to about 150 or more capture oligonucleotides.

In one aspect, one or more capture oligonucleotides include single stranded nucleic acid sequences, including for example, nucleic acid sequences including deoxyribonucleic acids (DNA), ribonucleic acids (RNA), or structural analogs that include non-naturally occurring chemical structures that can also participate in hybridization reactions.

In one aspect, the capture oligonucleotides used in a particular array have similar binding energies or melting temperatures (Tm), for example, within at least about 0.5° C., about 1° C., about 2° C., about 3° C., about 4° C., or about 5° C. of each other, wherein the melting temperature (Tₘ) of an oligonucleotide refers to the temperature at which 50% of the oligonucleotides is hybridized with its complement and 50% is free in solution. Tₘ can be determined using known methods, for example, by measuring the absorbance change of the oligonucleotide with its complement as a function of temperature. In one aspect, the capture oligonucleotide has a melting temperature (Tₘ) at 50 mM NaCl of between about 50° C. and about 70° C., about 55° C. and about 65° C., or at least about 50° C., about 55° C., or about 60° C. and up to about 60° C., about 65° C., or about 70° C. In one aspect, the capture oligonucleotide has a GC content between about 40% and about 60%, or about 40% and about 50%.

In one aspect, the capture oligonucleotide is between about 20 and about 100, about 30 and about 50, or about 35 and about 40 nucleotides in length, for example, at least about 20, about 21, about 22, about 23, about 24, about 25, about 26, about 27, about 28, about 29, about 30, about 31, about 32, about 33, about 34, about 35, or about 36 and up to about 36, about 37, about 38, about 39, about 40, about 41, about 42, about 43, about 44, about 45, about 46, about 47, about 48, about 49, about 50, about 75 or about 100 nucleotides in length. In one aspect, the capture oligonucleotide includes at least 20, about 24, about 30 or about 36 nucleotides. In one aspect, one or more capture oligonucleotides in an array are not identical in length to the nucleic acid sequence of its complementary oligonucleotide tag. In one aspect, the capture oligonucleotide has a sequence that is longer than the sequence of its complementary single stranded oligonucleotide tag, for example, by up to about 5, about 10, about 15, about 20 or about 25 bases.

In one aspect, one or more capture oligonucleotides are covalently or non-covalently immobilized to a support surface. In one aspect, one or more capture oligonucleotides are covalently or non-covalently immobilized to one or more binding domains on a support surface. In one aspect, the capture oligonucleotide is adsorbed to the support surface via electrostatic interactions, for example, between a negatively charged phosphate group on the oligonucleotide and a positive charge on the support surface. In one aspect, one or more capture oligonucleotides are immobilized to the support surface through the binding of a first binding partner attached (directly or through a linker moiety) to the capture oligonucleotide to a second binding partner that is immobilized on the surface. In one aspect, one or more capture oligonucleotides are covalently immobilized to the support surface. In one aspect, one or more capture oligonucleotides are directly immobilized to the support surface. In another aspect, the capture oligonucleotide is immobilized to the support surface through a linker.

Capture oligonucleotides are disclosed in International Application Publication No. WO 2020/227016, entitled “KITS FOR DETECTING ONE OR MORE TARGET NUCLEIC ACID ANALYTES IN A SAMPLE AND METHOD OF MAKING AND USING THE SAME” (Meso Scale Technologies, LLC., Rockville, MD, USA), the disclosure of which is incorporated by reference in its entirety.

### G. Support Surface

In one aspect, one or more capture oligonucleotides are immobilized on a support surface. The capture oligonucleotides can be immobilized on a variety of support surfaces, including support surfaces used in conventional binding assays. In one aspect, the support surface has a flat surface. In another aspect, the support surface has a curved surface. In one aspect, the support surface includes an assay module, such as an assay plate, slide, cartridge, bead, or chip. In one aspect, the support surface includes color coded microspheres. See, for example, Yang et al. (2001) BADGE, BeadsArray for the Detection of Gene Expression, a High-Throughput Diagnostic Bioassay. Genome Res. 11(11):1888-1898. In one aspect, the support surface includes one or more beads on which one or more capture oligonucleotides are immobilized.

Support surfaces can be made from a variety of suitable materials including polymers, such as polystyrene and polypropylene, ceramics, glass, composite materials, including, for example, carbon-polymer composites such as carbon-based inks. In one aspect, the support surface is a carbon-based support surface.

In one aspect, the support surface is provided by one or more particles or “beads”. In one aspect, the beads can have a diameter up to about 1 cm (or about 10,000 μm), about 5,000 μm, about 1,000 μm, about 500 μm or about 100 μm. In one aspect, beads have a diameter from about 10 nm and about 100 μm, from about 100 nm and about 10 μm or from about 0.5 μm and about 5 μm. In one aspect, the beads are paramagnetic, providing the ability to capture the beads through the use of a magnetic field. In one aspect, the support surface is provided by streptavidin or avidin-coated magnetic beads and biotin-labeled capture oligonucleotides are immobilized on the beads.

In one aspect, the support surface is a plate with a plurality of wells, i.e., a “multi-well plate.” Multi-well plates can include any number of wells of any size or shape, arranged in any pattern or configuration. In one aspect, the multi-well plate includes from about 1 to about 10,000 wells. In one aspect, the multi-well assay plates use industry standard formats for the number, size, shape and configuration of the plate and wells. Examples of standard formats include 96-, 384-, 1536- and 9600-well plates, with the wells configured in two-dimensional arrays. Other multi-well formats include single well, two well, six well and twenty-four well and 6144 well plates. In one aspect, the support surface includes a 96 well-plate.

In one aspect, the support surface includes a two-dimensional patterned array in which capture oligonucleotides are printed at known locations, referred to as binding domains. In one aspect, the support surface includes a patterned array of discrete, non-overlapping, addressable binding domains to which capture oligonucleotides are immobilized, wherein the sequence of the capture oligonucleotide in each binding domain is known and can be correlated with an appropriate target analyte. In one aspect, all capture oligonucleotides in a particular binding domain have the same sequence and the capture oligonucleotides in one binding domain have a sequence different from capture oligonucleotides in other binding domains. In one aspect, multiple binding domains are arrayed in orderly rows and columns on a support surface and the precise location and sequence of each binding domain is recorded in a computer database. In one aspect, the array is arranged in a symmetrical grid pattern. In other aspects, the array is arranged another pattern, including, but not limited to, radially distributed lines, spiral lines, or ordered clusters. In another aspect, each binding domain is positioned on a surface of one or more microparticles or beads wherein the microparticles or beads are coded to allow for discrimination between different binding domains.

In one aspect, the support surface is a multi-well plate that includes one or more discrete addressable binding domains within each well that correspond to one or more capture oligonucleotides. In one aspect, the support surface includes at least one binding domain for detecting a wild-type nucleotide sequence and separate binding domain for detecting a mutant nucleotide sequence. In one aspect, each well includes at least about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24 or about 25 binding domains. In one aspect, each well includes at least about 7, about 10, about 16, or about 25 binding domains.

In one aspect, the support surface is a multi-well plate that includes at least 24, 96, or 384 wells and each well includes array of up to 10 binding domains in which different capture oligonucleotides are immobilized in discrete binding domains. In a more particular aspect, the support surface is a 96 well plate in which each well includes an array having up to 10 binding domains. In one aspect, each well of a 96-well plate includes up to 10 binding domains, having up to 10 distinct capture oligonucleotides immobilized thereon. In one aspect, each well includes the same patterned array with the same capture oligonucleotides. In another aspect, different wells may include a different patterned array of capture oligonucleotides.

### H. Nuclease Protection Assay (NPA)

In one aspect, a method is provided for detecting or quantifying a sense and an antisense strand of an oligonucleotide duplex in a sample using a nuclease protection assay. In one aspect, the sample is contacted with a set of probes, wherein the set of probes includes a sense probe and an antisense probe. In one aspect, the sense probe includes a single stranded oligonucleotide tag that is complementary to at least a portion of a capture oligonucleotide immobilized on a support surface, a sense binding portion capable of hybridizing to a nucleotide sequence of the sense strand of the oligonucleotide duplex, and a label. In one aspect, the antisense probe includes a single stranded oligonucleotide tag that is complementary to at least a portion of a capture oligonucleotide immobilized on the support surface, an antisense binding portion capable of hybridizing to a nucleotide sequence of the antisense strand of the oligonucleotide duplex, and a label. In one aspect, the method includes incubating the probes with the sample to form a hybridization mixture that contains hybridization complexes. In one aspect, the method includes contacting a support surface with the hybridization mixture under conditions in which the oligonucleotide tags of the hybridization complexes hybridize to the capture oligonucleotides immobilized on the support surface. In one aspect, the method includes detecting or quantifying the sense and antisense strands of the oligonucleotide duplex based on the presence of label immobilized on the support surface. In one aspect, the method includes detecting or quantifying the sense and antisense strands of the oligonucleotide duplex based on the presence of labelled sense and antisense hybridization complexes immobilized on the support surface.

In one aspect, the sense binding portion of the sense probe has a sense binding length that is shorter than a sense strand length of the sense strand. In one aspect, the sense binding portion of the sense probe has a sense binding length that is shorter than a sense strand length of the sense strand by at least 1 nucleotide. In one aspect, the antisense binding portion of the antisense strand has an antisense binding length that is shorter than an antisense strand length of the antisense strand. In one aspect, the antisense binding portion of the antisense strand has an antisense binding length that is shorter than an antisense strand length of the antisense strand by at least 1 nucleotide.

In one aspect, the sense probe includes DNA. In one aspect, the sense binding portion of the sense probe includes DNA. In one aspect, the oligonucleotide tag of the sense probe includes DNA. In one aspect, the sense binding portion and the oligonucleotide tag of the sense probe include DNA.

In one aspect, the sense probe includes RNA. In one aspect, the sense binding portion of the sense probe includes RNA. In one aspect, the oligonucleotide tag of the sense probe includes RNA. In one aspect, the sense binding portion and the oligonucleotide tag of the sense probe include RNA.

In one aspect, the sense binding portion of the sense probe includes DNA and the oligonucleotide tag of the sense probe includes RNA. In one aspect, the sense binding portion of the sense probe includes RNA and the oligonucleotide tag of the sense probe includes DNA.

In one aspect, the antisense probe includes DNA. In one aspect, the antisense binding portion of the antisense probe includes DNA. In one aspect, the oligonucleotide tag of the antisense probe includes DNA. In one aspect, the antisense binding portion and the oligonucleotide tag of the antisense probe include DNA.

In one aspect, the antisense probe includes RNA. In one aspect, the antisense binding portion of the antisense probe includes RNA. In one aspect, the oligonucleotide tag of the antisense probe includes RNA. In one aspect, the antisense binding portion and the oligonucleotide tag of the antisense probe include RNA.

In one aspect, the antisense binding portion of the antisense probe includes DNA and the oligonucleotide tag of the antisense probe includes RNA. In one aspect, the antisense binding portion of the antisense probe includes RNA and the oligonucleotide tag of the antisense probe includes DNA.

In one aspect, the antisense probe is a chimeric probe that includes an antisense binding portion that includes DNA and an oligonucleotide tag that includes RNA, wherein the antisense binding portion the antisense probe has an antisense binding length that is shorter than the antisense strand length of the antisense strand of the oligonucleotide duplex by at least 1 nucleotide. In one aspect, the antisense probe is a chimeric probe that includes an antisense binding portion that includes RNA and an oligonucleotide tag that includes DNA, wherein the antisense binding portion the antisense probe has an antisense binding length that is shorter than the antisense strand length of the antisense strand of the oligonucleotide duplex by at least 1 nucleotide. In one aspect, the sense probe is a chimeric probe that includes a sense binding portion that includes DNA and an oligonucleotide tag that includes RNA, wherein the sense binding portion the sense probe has a sense binding length that is shorter than the sense strand length of the sense strand of the oligonucleotide duplex by at least 1 nucleotide. In one aspect, the sense probe is a chimeric probe that includes a sense binding portion that includes RNA and an oligonucleotide tag that includes DNA, wherein the sense binding portion the sense probe has a sense binding length that is shorter than the sense strand length of the sense strand of the oligonucleotide duplex by at least 1 nucleotide.

In one aspect, the method includes incubating the set of probes with the sample to form a hybridization mixture. In one aspect, the hybridization mixture contains hybridization complexes that include a sense complex and an antisense complex. In one aspect, the hybridization mixture includes a sense complex that includes the sense probe hybridized with the sense strand of the oligonucleotide duplex; and an antisense complex that includes the antisense probe hybridized with the antisense strand of the oligonucleotide duplex.

In one aspect, the hybridization mixture further contains one or more of the following unproductive hybridization complexes: a sense probe that is not hybridized with a sense strand of the oligonucleotide duplex; an antisense probe that is not hybridized to the antisense strand of the oligonucleotide duplex; or a probe-probe complex in which the sense probe and antisense probes hybridize to each other. In one aspect, the unproductive hybridization complex includes a single strand overhang. In one aspect, the unproductive hybridization complex includes a single strand oligonucleotide sequence. In one aspect, the unproductive hybridization complex includes a single strand RNA sequence. In one aspect, the unproductive hybridization complex includes a single strand DNA sequence.

In one aspect, the single-strand overhang or single strand oligonucleotide sequence in an unproductive hybridization complex is digested with a single-strand specific nuclease. In one aspect, the single-strand overhang or single strand oligonucleotide sequence is a DNA sequence that is digested with a single-strand specific DNase. In one aspect, the single-strand overhang or single strand oligonucleotide sequence is an RNA sequence that is digested with a single-strand specific RNase.

In one aspect, digestion of the single-stranded overhang or single strand oligonucleotide sequence in an unproductive hybridization complex is performed while the hybridization complexes are in solution (i.e., before they hybridization complexes are immobilized to the support surface via the oligonucleotide tags of the sense or antisense probes). In one aspect, digestion of the single-stranded overhang or single strand oligonucleotide sequence in an unproductive hybridization complex is performed after the hybridization complexes are immobilized on a support surface via the oligonucleotide tags of the sense or antisense probes. In one aspect, the method includes a wash step after the single stranded overhang or single stranded oligonucleotide sequences are digested with a single-strand specific RNase In one aspect, the method includes a wash step after the single stranded overhang or single stranded oligonucleotide sequences are digested with a single-strand specific RNase and immobilized on the support surface. In one aspect, the sense and antisense strands of the oligonucleotide duplex are detected or quantified based on the presence of label immobilized on the support surface. In one aspect, the sense and antisense strands of the oligonucleotide duplex are detected or quantified based on the presence of labeled sense or antisense complexes immobilized on the support surface.

In one aspect, the method provided herein comprises an RNase treatment and incubation step during hybridization, or after the hybridization complexes are immobilized on a support surface via the oligonucleotide tags of the sense or antisense probes, or both. In one aspect, the RNase treatment is performed at a temperature of about 25° C. to about 40° C. In one aspect, the RNase treatment is performed at a temperature of less than about 37° C. In one aspect, the RNase treatment is performed at a temperature of about 27° C. to about 37° C. In one aspect, the RNase treatment is performed at a temperature of about 25° C., 30° C., or 37° C. Preferably, the RNase treatment is performed at a temperature of about 30° C. In one aspect, the RNase incubation time is about 30 to about 60 minutes or about 30 to 90 minutes. In one aspect, the RNase incubation time is about 30 minutes. Preferably, the RNase incubation time is about 60 minutes.

In one aspect, use of a lysis buffer during RNase treatment and incubation is provided herein. In one aspect, the lysis buffer comprises sodium dodecyl sulphate (SDS), lithium dodecyl sulphate (SDS), Triton X (100, 114), NP-40, Tween (20, 80), Cetyltrimethylammonium bromide (CTAB), CHAPS, CHAPSO, Proteinase K, or any combination thereof. It is to be understood that any lysis buffer known to one of ordinary skill in the art can be used in addition to the provided lysis buffers. In one aspect, the lysis buffer includes a diluent, lithium dodecyl sulfate (LDS), and/or Proteinase K. In one aspect, the lysis buffer includes a diluent, 2% lithium dodecyl sulfate (LDS), and 0.4 mg/mL Proteinase K.

### I. Electrodes

In one aspect, a sense or an antisense strand of an oligonucleotide duplex is detected or quantified using electrochemiluminescence (ECL). Multiplexed measurement of analytes using electrochemiluminescence is described in U.S. Pat. Nos. 7,842,246 and 6,977,722, the disclosures of which are incorporated herein by reference in their entireties.

In one aspect, the support surface includes one or more electrodes. In one aspect, the support surface includes one or more working electrodes and one or more counter electrodes. In one aspect, the support surface includes one or more binding domains formed on one or more electrodes for use in electrochemical or electrochemiluminescence assays.

In one aspect, the binding domains are formed by collecting beads coated with capture oligonucleotides onto the electrode surface. In one aspect, the beads are paramagnetic and the beads are collected on the electrode through the use of a magnetic field.

In one aspect, the electrodes are provided within an assay module that provides assay containers, assay flow cells, assay fluidics or other components useful for carrying out an assay. Examples of assay modules for carrying out electrochemiluminescence assays include, for example, multiarray case, assay plates case, cartridge case, and the like. In one aspect, the electrodes are provided within an assay module that provides assay containers, assay flow cells, assay fluidics or other components useful for carrying out an assay. Examples of assay modules for carrying out electrochemiluminescence assays can be found in U.S. Pat. Nos. 6,673,533, 7,842,246, 9,731,297, and 8,298,834. In one aspect, the support surface is multi-well plate that includes at least one electrode. In one aspect, each well of a multi-well assay plate includes at least one electrode. In one aspect, at least one well of the multi-well assay plate includes a working electrode. In another aspect, at least one well of the multi-well assay plate includes a working electrode and a counter electrode. In another aspect, each well of the multi-well assay plate includes a working electrode and a counter electrode. In one aspect, the working electrode is adjacent, but not in electrical contact with the counter electrode.

In one aspect, the electrodes are constructed from a conductive material, including, for example, a metal such as gold, silver, platinum, nickel, steel, iridium, copper, aluminum, a conductive alloy, or combinations thereof. In another aspect, the electrodes include semiconducting materials such as silicon and germanium or semi-conducting films such as indium tin oxide (ITO) and antimony tin oxide (ATO). In another aspect, the electrodes include oxide coated metals, such as aluminum oxide coated aluminum. In one aspect, the electrode includes a carbon-based material. In one aspect the electrodes include mixtures of materials containing conducting composites, inks, pastes, polymer blends, and metal/non-metal composites, including for example, mixtures of conductive or semi-conductive materials with non-conductive materials. In one aspect, the electrodes include carbon-based materials such as carbon, glassy carbon, carbon black, graphitic carbon, carbon nanotubes, carbon fibrils, graphite, carbon fibers and mixtures thereof. In one aspect, the electrodes include conducting carbon-polymer composites, conducting polymers, or conducting particles dispersed in a matrix, for example, carbon inks, carbon pastes, or metal inks. In one aspect, the working electrode is made of a carbon-polymer composite that includes, for example, conducting carbon particles, such as carbon fibrils, carbon black, or graphitic carbon, dispersed in a matrix, for example, a polymer matrix such as ethylene vinyl acetate (EVA), polystyrene, polyethylene, polyvinyal acetate, polyvinyl chloride, polyvinyl alcohol, acrylonitrile butadiene styrene (ABS), or copolymers of one or more of these polymers.

In one aspect, the working electrode is made of a continuous conducting sheet or a film of one or more conducting materials, which may be extruded, pressed or molded. In another aspect, the working electrode is made of a conducting material deposited or patterned on a substrate, for example, by printing, painting, coating, spin-coating, evaporation, chemical vapor deposition, electrolytic deposition, electroless deposition, photolithography or other electronics microfabrication techniques. In one aspect, the working electrode includes a conductive carbon ink printed on a polymeric support, for example, by ink-jet printing, laser printing, or screen-printing. Carbon inks are known and include materials produced by Acheson Colloids Co. (e.g., Acheson 440B, 423ss, PF407A, PF407C, PM-003A, 30D071, 435A, Electrodag 505SS, and Aquadag™), E. I. Du Pont de Nemours and Co. (e.g., Dupont 7105, 7101, 7102, 7103, 7144, 7082, 7861D, and CB050), Conductive Compounds Inc (e.g., C-100), and Ercon Inc. (e.g., G-451).

In one aspect, the working electrode is a continuous film. In another aspect, the working electrode includes one or more discrete regions or a pattern of discrete regions. Alternately, the working electrode may include a plurality of connected regions. One or more regions of exposed electrode surface on a working electrode can be defined by a patterned insulating layer covering the working electrode, for example, by screen printing a patterned dielectric ink layer over a working electrode, or by adhering a die-cut insulating film. The exposed regions may define the array elements of arrays of reagents printed on the working electrode and may take on array shapes and patterns as described above. In one aspect, the insulating layer defines a series of circular regions (or “spots”) of exposed working electrode surface.

A counter electrode may have one or more of the properties described above generally for working electrodes. In one aspect, the working and counter electrodes are constructed from the same material. In another aspect, the working and counter electrodes are not constructed from the same material, for example, the working electrode may be a carbon electrode and the counter electrode may be a metal electrode.

In one aspect, one or more capture oligonucleotides are immobilized on one or more electrodes by passive adsorption. In another aspect, one or more capture oligonucleotides are covalently immobilized on the electrodes. In one aspect the electrodes are derivatized or modified, for example, to immobilize reagents such as capture oligonucleotides on the surface of the electrodes. In one aspect, the electrode is modified by chemical or mechanical treatment to improve the immobilization of reagents, for example, to introduce functional groups for immobilization of reagents or to enhance its adsorptive properties. Examples of functional groups that can be introduced include, but are not limited, to carboxylic acid (COOH), hydroxy (OH), amino (NH₂), activated carboxyls (e.g., N-hydroxy succinimide (NHS)-esters), poly-(ethylene glycols), thiols, alkyl ((CH₂)ₙ) groups, or combinations thereof). In one aspect, one or more reagents, for example, capture oligonucleotides, are immobilize by either covalent or non-covalent means to a carbon-containing electrode, for example, carbon black, fibrils, or carbon dispersed in another material. It has been found that capture oligonucleotides having thiol groups can bind covalently to carbon-containing electrodes, for example to screen-printed carbon ink electrodes, without having to first deposit an additional thiol-reactive layer such as a protein layer or a chemical cross-linking layer. In one aspect, methods are provided for direct attachment of capture oligonucleotides having thiol groups, such as thiol-modified oligonucleotides, to electrodes which provide simple, robust, efficient and reproducible processes for forming capture surfaces and arrays on electrodes. In one aspect, one or more capture oligonucleotides having thiol groups are directly immobilized on carbon-containing electrodes, such as screen-printed carbon ink electrodes, through reaction of the thiols with the electrode, without first adding a thiol-reactive layer to the electrode.

In one aspect the electrode is treated with a plasma, for example, a low temperature plasma, such as a glow-discharge plasma, to alter the physical properties, chemical composition, or surface-chemical properties of the electrode, for example, to aid in the immobilization of reagents such as a capture oligonucleotide, or to reduce contaminants, improve adhesion to other materials, alter the wettability of the surface, facilitate deposition of materials, create patterns, or improve uniformity. Examples of useful plasmas include oxygen, nitrogen, argon, ammonia, hydrogen, fluorocarbons, water and combinations thereof. In one aspect, oxygen plasma is used to treat an electrode with carbon particles in a carbon-polymer composite material. In another aspect, oxygen is used to introduce carboxylic acids or other oxidized carbon functionality into carbon or organic materials (for example, activated esters or acyl chlorides) to facilitate coupling of reagents. In another aspect, ammonia-containing plasmas may be used to introduce amino groups for use in coupling assay reagents. In one aspect, the electrode is not pretreated to aid in the immobilization of one or more capture oligonucleotides.

In one aspect, the support surface includes an assay module such as a multi-well plate having one or more working or counter electrodes in each well. In one aspect, the multi-well plate includes a plurality of working or counter electrodes in each well. In one aspect, the working or counter electrodes of the multi-well plate include carbon, for example, screen-printed layers of carbon inks. In one aspect, one or more capture oligonucleotides are immobilized on the screen-printed carbon ink through a thiol moiety on the capture oligonucleotide. In one aspect, the working electrode is used to induce an ECL signal from an ECL label. In one aspect, the ECL signal is emitted from ruthenium-tris-bipyridine in the presence of a co-reactant such as a tertiary alkyl amine, for example, tripropyl amine or butyldiethanolamine.

In one aspect, the electrode contains binding domains as described above that are defined by dielectric ink (i.e., electrically insulating ink). The electrode is a working electrode with a dielectric printed over it in a pattern that defines the binding domains described above. In one aspect, the binding domains are roughly circular areas of exposed working electrode (or “spots”). The electrodes are in 96-well plates formed by adhering an injection molded 96-well plate top to a mylar sheet that defines the bottom of the wells. The top surface of the mylar sheet has screen printed carbon ink electrodes printed on it such that each well includes a carbon ink working electrode roughly in the center of the well and two carbon ink counter electrodes roughly towards two edges of the well. The electrodes printed on the bottom of the mylar sheet, connected through conductive through-holes to the top of the sheet, provide contacts for applying electrical voltage to the working and counter electrodes.

### J. Detection

In one aspect, the presence of one or more oligonucleotides sequences is detected or quantified based on the detection of a label immobilized on the support surface. In one aspect, the presence of one or more oligonucleotide sequences is detected or quantified based on the detection of a label on a hybridization complex immobilized on the support surface. In one aspect, the presence of one or more target oligonucleotide sequences is detected or quantified based on the detection of a label on a sense complex immobilized on the support surface. In one aspect, the presence of one or more target oligonucleotide sequences is detected or quantified based on the detection of a label on an antisense complex immobilized on the support surface. In one aspect, an oligonucleotide sequence is detected or quantified in an array. In one aspect, the sense and antisense strands of an oligonucleotide sequence are detected in a well of, e.g., a multi-well plate. In one aspect, the antisense and sense strands of the same oligonucleotide sequence are detected in the same well. In one aspect, the antisense and sense strands of the same oligonucleotide sequence are detected in separate wells.

In one aspect, the presence of the hybridization complex, for example, the antisense or sense complex is detected by monitoring light emission from a label, including, but not limited to, fluorescence, time-resolved fluorescence, fluorescence resonance energy transfer (FRET), fluorescence polarization (FP), luminescence, chemiluminescence, bioluminescence, phosphorescence, light scattering or electrode induced luminescence. In another aspect, the label includes enzymes or other chemically reactive species with a chemical activity that leads to a measurable signal such as light scattering, absorbance, fluorescence, etc. Examples of enzyme labels include, but are not limited to, horseradish peroxidase or alkaline phosphatase. In one aspect, the label is a detectable hapten, including, but not limited to, biotin, fluorescein or digoxigenin. In one aspect, the label includes biotin.

In one aspect, the hybridization complex, for example, the antisense complex or sense complex is immobilized on one or more binding domains located on the support surface. In one aspect, one or more binding domains are located on one or more electrodes and detecting or quantifying includes applying a voltage waveform to one or more electrodes to stimulate the labels on the captured reaction products to produce an electrochemical or luminescent signal. In one aspect, detecting or quantifying includes measuring an ECL signal and correlating the signal with the presence or an amount of sense or antisense oligonucleotide in a sample. In one aspect, the intensity of the emitted light is proportional to the amount of sense or antisense oligonucleotide in the sample such that the emitted light can provide a quantitative determination of the amount of sense or antisense oligonucleotide in the sample.

In one aspect, the support surface is contacted with a detection mixture after the hybridization complexes are immobilized thereon. In one aspect, the support surface is contacted with a detection mixture after the sense or antisense complexes are immobilized thereon. In one aspect, the support surface is contacted with a detection mixture after the single-strand overhang in any unproductive hybridization complexes is digested with a single-stranded nuclease. In one aspect, digestion of the single-stranded overhang in an unproductive hybridization complex is performed while the hybridization complexes are in solution (i.e., before they hybridization complexes are immobilized to the support surface via the oligonucleotide tags of the sense or antisense probes). In one aspect, digestion of the single-stranded overhang in an unproductive hybridization complex is performed after the hybridization complexes are immobilized on the support surface via the oligonucleotide tags of the sense or antisense probes. In one aspect, the detection mixture includes an ECL label. Examples of ECL labels include: i) organometallic compounds where the metal is from, for example, the noble metals of group VIII, including Ru-containing and Os-containing organometallic compounds such as the tris-bipyridyl-ruthenium (RuBpy) moiety and ii) luminol and related compounds. In one aspect, the detection mixture also includes one or more electrochemiluminescence co-reactants, and one or more additional components such as a pH buffering agent, detergent, preservative, anti-foaming agent, salt, metal ion or metal chelating agent. The term “electrochemiluminescent co-reactant” refers to species that participate with the electrochemiluminescent label to and include, but are not limited to, tertiary amines, such as tripropylamine (TPA), oxalate ion, ascorbic acid and persulfate for RuBpy and hydrogen peroxide for luminol. Methods for measuring electrochemiluminescence are known and instruments for making the measurements are commercially available. For example, multiplexed measurement of analytes using electrochemiluminescence is used in the Meso Scale Diagnostics, LLC, MULTI-ARRAY® and SECTOR® Imager line or products (see, e.g., U.S. Pat. Nos. 7,842,246 and 6,977,722, the disclosures of which are incorporated herein by reference in their entireties).

In one aspect, biotin is covalently attached to the hybridization complex and the detection mixture includes a streptavidin-conjugated label which binds to the immobilized hybridization complex through the avidin moiety. In one aspect, the streptavidin-conjugated label is an electrochemiluminescent (ECL) label. In one aspect, the electrochemiluminescent label is an n-hydroxysuccinimide ester, such as the Sulfo-TAG NHS-Ester (Meso Scale Diagnostics, Rockville, MD, U.S.A.).

### K. Kit

In one aspect, a kit is provided for carrying out the method described herein. “Kit” refers to a set of components that are provided or gathered to be used together, for example, to create a composition, to manufacture a device, or to carry out a method. A kit can include one or more components. The components of a kit may be provided in one package or in multiple packages, each of which can contain one or more of the components. A listed component of a kit, may in turn, also be provided as a single physical part or as multiple parts to be combined for the kit use. For example, an instrument component of a kit may be provided fully assembled or as multiple instrument parts to be assembled prior to use. Similarly, a liquid reagent component of a kit may be provided as a complete liquid formulation in a container, as one or more dry reagents and one or more liquid diluents to be combined to provide the complete liquid formulation, or as two or more liquid solutions to be combined to provide the complete liquid formulation. As is known in the art, kit components for assays are often shipped and stored separately due to having different storage needs, e.g., storage temperatures of 4° C. versus −70° C.

In one aspect, the kit includes a support surface. In one aspect, the kit includes capture oligonucleotides that can be immobilized on the support surface. In one aspect, the kit includes capture oligonucleotides immobilized on a support surface. In one aspect, the kit includes capture oligonucleotides immobilized on a support surface in an array. In one aspect, the kit includes one or more capture oligonucleotides immobilized to one or more discrete binding domains with a known location within an array. In one aspect, the kit includes two or more capture oligonucleotides immobilized on a bead array.

In one aspect, the kit includes a carbon-based support surface. In one aspect, the support surface includes at least one electrode. In one aspect, the electrode is a carbon-based electrode. In one aspect, the support surface includes one or more carbon ink electrodes. In one aspect, the support surface includes at least one working electrode and at least one counter electrode.

In one aspect, the kit includes a support surface that includes a multi-well assay plate. In one aspect, one or more wells of the multi-well plate include one or more electrodes. In one aspect, the support surface includes a multi-well plate wherein one or more wells include one or more working electrodes and one or more counter electrodes. In one aspect, the support surface includes one or more reference electrodes.

In one aspect, the kit includes a standard format multi-well plate, which are known in the art and can include, but are not limited to, 24, 96, and 384 well plates. In one aspect, the kit includes one or more 96 well plates. In one aspect, the kit includes one multi-well plate. In another aspect, the kit includes 10 multi-well plates. In another aspect, the kit includes from 10 and 100 multi-well plates.

In one aspect, the kit includes a support surface having one or more electrodes on which one or more arrays of capture oligonucleotides are printed. In one aspect, the kit includes one or more multi-well plates on which one or more arrays of capture oligonucleotides have been printed. In another aspect, the kit includes one or more multi-well plates and one or more vials that include one or more capture oligonucleotides, wherein the capture oligonucleotides can be printed onto the multi-well plates.

In one aspect, the kit includes one or more capture oligonucleotides immobilized to one or more binding domains on the support surface. In one aspect, the kit includes one or more capture oligonucleotides immobilized on one or more binding domains within a well of a multi-well plate. In one aspect, the kit includes one or more capture oligonucleotides immobilized on one or more binding domains on an electrode. In one aspect, the kit includes one or more capture oligonucleotides immobilized on one or more binding domains on an electrode within one or more wells of a multi-well plate.

In another aspect, the kit includes one or more oligonucleotide tags. In one aspect, the kit includes one or more oligonucleotide tags provided in containers, wherein the oligonucleotide tags in a container have the same sequence and each container contains oligonucleotide tags having a sequence different from (and not complementary to) the sequence of the oligonucleotide tags in the other containers. In one aspect, the kit includes, in separate containers, at least about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24 or about 25 and up to about 64 unique oligonucleotide tags. In one aspect, the kit includes a set of up to 10 unique oligonucleotide tags.

In one aspect, the kit includes one or more multi-well plates in which up to 10 capture oligonucleotides are immobilized in one or more binding domains within a well of a multi-well plate, wherein each binding domain includes a capture oligonucleotide having a sequence that is different than the sequences of the capture oligonucleotides in the other binding domains within the well. In one aspect, the kit includes a support surface having at least about 1, about 2, about 3, about 4, about 5, about 6, about 7, about 8, about 9, about 10, about 15, about 20, or about 25 distinct capture oligonucleotides immobilized in at least about 1, about 2, about 3, about 4, about 5, about 6, about 7, about 8, about 9, about 10, about 15, about 20, or about 25 unique binding domains. In one aspect, the kit includes a multi-well plate having at least about 1, about 2, about 3, about 4, about 5, about 6, about 7, about 8, about 9, about 10, about 15, about 20, or about 25 distinct capture oligonucleotides immobilized in at least about 1, about 2, about 3, about 4, about 5, about 6, about 7, about 8, about 9, about 10, about 15, about 20, or about 25 unique binding domains in one or more wells. In one aspect, the kit includes one or more multi-well plates in which each well includes up to about 10 capture oligonucleotides immobilized in an array. In one aspect, the multi-well plate can be configured to create from about 1 and about 10 detection assays within each well of the multi-well plate.

In one aspect, the kit includes a single-stranded nuclease. In one aspect, the kit includes a single-stranded nuclease tags provided in a container. In one aspect, the single-strand specific nuclease includes a single-strand specific DNase. In one aspect, the single-strand specific DNase is S1 nuclease, P1 nuclease or Mung Bean nuclease. In one aspect, the single-strand specific nuclease includes a single-strand specific RNase. In one aspect, the single-strand specific RNase is RNase A, RNase H, RNase I, RNase III, RNase L, RNase P, RNase PhyM, RNase T1, RNase T2, RNase U2, RNase V, PNPase, RNase PH, RNase R, RNase D, RNase T, RNaseONE, oligoribonuclease, exoribonuclease I, or exoribonuclease II. In one aspect, the kit disclosed herein comprises a lysis buffer. In one aspect, the lysis buffer comprises sodium dodecyl sulphate (SDS), lithium dodecyl sulphate (SDS), Triton X (100, 114), NP-40, Tween (20, 80), Cetyltrimethylammonium bromide (CTAB), CHAPS, CHAPSO, Proteinase K, or any combination thereof. It is to be understood that any lysis buffer known to one of ordinary skill in the art can be used in addition to the provided lysis buffers. In one aspect, the lysis buffer includes a diluent, lithium dodecyl sulfate (LDS), and/or Proteinase K. In one aspect, the lysis buffer includes a diluent, 2% lithium dodecyl sulfate (LDS), and 0.4 mg/mL Proteinase K.

In one aspect, a kit is provided for conducting a luminescence assay, for example, an electrochemiluminescence assay to detect or quantify one or more target nucleotide sequences in a sample. In one aspect, the kit includes one or more assay components useful in carrying out an electrochemiluminescence assay.

In one aspect, the kit includes hybridization buffer that can be used to provide the appropriate conditions (e.g., stringent conditions) for hybridization of oligonucleotide tags to their corresponding complementary capture oligonucleotides sequences. In one aspect, the hybridization buffer includes Diluent 54 (Meso Scale Diagnostics, LLC, Rockville, MD, U.S.A.). In one aspect, the hybridization buffer includes Hybridization Buffer 1 or Hybridization Buffer 2 (Meso Scale Diagnostics, LLC, Rockville, MD, U.S.A.).

In one aspect, the kit includes one or more containers that include a label. In one aspect, the label is selected from a radioactive, fluorescent, chemiluminescent, electrochemiluminescent, light absorbing, light scattering, electrochemical, magnetic and an enzymatic label. In one aspect, the label includes an electrochemiluminescent label. In one aspect, the label includes an organometallic complex that includes a transition metal. In one aspect, the transition metal includes ruthenium. In one aspect, the label is a MSD SULFO-TAG™ label (Meso Scale Diagnostics, LLC, Rockville, MD, U.S.A.).

In one aspect, the label includes a primary binding reagent that is a binding partner of a secondary binding reagent. In one aspect, the secondary binding reagent includes biotin, streptavidin, avidin, or an antibody. In one aspect, the secondary binding reagent includes avidin, streptavidin or an antibody. In one aspect, the label includes a hapten selected from biotin, fluorescein and digoxigenin. In one aspect, the label is a primary binding agent that includes a first oligonucleotide sequence and the secondary binding reagent includes a second oligonucleotide sequence that is complementary to the first oligonucleotide sequence of the primary binding agent.

In one aspect, the kit includes one or more containers that include an electrochemiluminescent label. In a more particular aspect, the kit includes one or more containers containing Ru-containing or Os-containing organometallic compounds such as tris-bipyridyl-ruthenium (RuBpy). In one aspect, the label includes an organometallic complex that includes a transition metal. In one aspect, the transition metal includes ruthenium. In one aspect, the label includes the MSD SULFO-TAG™ label (Meso Scale Diagnostics, LLC, Rockville, MD, U.S.A.). In another aspect, the kit includes one or more containers containing luminol or other related compounds.

In one aspect, the kit includes one or more containers with one or more electrochemiluminescent co-reactants. In one aspect, one or more electrochemiluminescent co-reactants are covalently or non-covalently immobilized on the support surface. In one aspect, one or more electrochemiluminescent co-reactants are immobilized on one or more working electrodes of the support surface.

In one aspect, the label included in the kit includes a primary binding reagent and a secondary binding reagent. In one aspect, the secondary binding reagent includes biotin, streptavidin, avidin or an antibody.

In one aspect, the kit includes one or more of the following assay components: one or more capture oligonucleotides; and one or more buffers, for example, a wash buffer, a hybridization buffer, a binding buffer, or a read buffer.

In one aspect, the kit includes one or more assay components such as a label. In one aspect, the label is a luminescent label such as an electrochemiluminescent label. In one aspect, the kit includes at least one electrochemiluminescence co-reactant. In one aspect, the electrochemiluminescent co-reactant includes a tertiary amine, tripropylamine, or N-butyldiethanolamine.

In one aspect, the kit includes one or more other assay components. In one aspect, the kit includes one or more assay including, but not limited to, a diluent, blocking agents, stabilizing agents, detergents, salts, pH buffers, and preservatives. In one aspect, the kit includes containers of one or more such components. In another aspect, one or more reagents are included on the assay support surface provided with the kit.

### L. Incorporation by Reference

All references cited herein, including patents, patent applications, papers, text books and the like, and the references cited therein, to the extent that they are not already, are hereby incorporated herein by reference in their entirety for all purposes.

### WORKING EXAMPLES

### Example 1 siRNA Detection: Use of Lysis Buffer

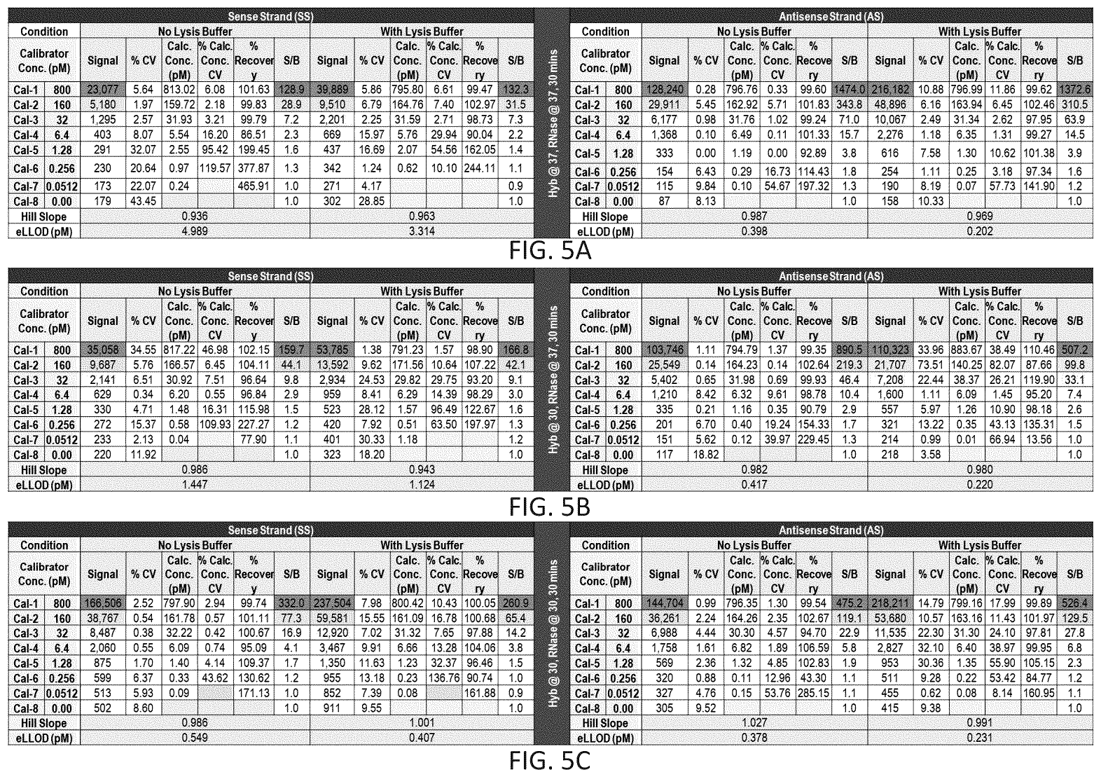

Briefly, individual antisense (AS) or sense (SS) strands of an siRNA were prepared and a 8-point calibration curve was generated for the individual antisense or sense strands by spiking the siRNA into Diluent 54 with a highest calibrator concentration of 800 pM using 5-fold serial dilutions as shown in FIG. 5A.

The hybridization protocol for the sense (SS) and antisense (AS) strands of a model siRNA oligonucleotide was performed as outlined in Table 1.

TABLE 1
Hybridization Protocol

| Step | Temp. | Time | |
|---|---|---|---|
| 1 | 98° C. | 2 min. | |
| 2 | 65° C. | About 30 | Gradually decrease |
| | | mins. | temperature to 4° C. at 50% |
| | | | ramp rate. |
| 3 | 4° C. | Hold | Hold at 4° C. |

A 96-well N-PLEX® plate (Meso Scale Discovery, Rockville, MD, USA (“MSD”)), on which single stranded capture oligonucleotides (with sequences complementary to the oligonucleotide tag sequences of the chimeric sense and antisense probes) were immobilized, was blocked with N-PLEX™ blocking buffer (MSD) and washed 3× with a minimum of 150 L/well Dulbecco's phosphate-buffered saline (DPBS). After the probes were hybridized to their target strands, the probe/analyte complexes were diluted in buffer, added to the plate and allowed to hybridize for 1 hour at 37° C., while shaking.

The plates were washed again (3× with a minimum of 150 μL/well DPBS) and an RNase cocktail (RNase A, and RNase T1) in Diluent 54 or Lysis Buffer (MSD Diluent 54+2% LDS+0.4 mg/mL Proteinase K.) was added to the plate and incubated for 30 min at either 30° C. or 37° C., while shaking.

The ECL signals for the SS and AS strands are shown in FIG. 5A. The probes against both strands had a higher ECL reading with use of the lysis buffer than the probes against both strands in Diluent 54 only. The results in FIG. 5A demonstrate higher sensitivity of detection of both SS and AS strands of the siRNA with the addition of lysis buffer.

### Example 2 siRNA Detection: Hybridization Treatment at 30° C.

Briefly, individual antisense (AS) or sense (SS) strands of an siRNA were prepared and a 8-point calibration curve was generated for the individual antisense or sense strands by spiking the siRNA into Diluent 54 with a highest calibrator concentration of 800 pM using 4-fold serial dilutions as shown in FIG. 5B.

The detection limits for the sense (SS) and antisense (AS) strands of a model siRNA oligonucleotide (ASO) were determined at two different hybridization temperatures.

A 96-well N-PLEX® plate (Meso Scale Discovery, Rockville, MD, USA (“MSD”)), on which single stranded capture oligonucleotides (with sequences complementary to the oligonucleotide tag sequences of the chimeric sense and antisense probes) were immobilized, was blocked with N-PLEX™ blocking buffer (MSD) and washed 3× with a minimum of 150 μL/well Dulbecco's phosphate-buffered saline (DPBS). After the probes were hybridized to their target strands, the probe/analyte complexes were diluted in buffer, added to the plate and allowed to hybridize for 1 hour at either 30° C. or 37° C., while shaking.

The plates were washed again (3× with a minimum of 150 μL/well DPBS) and an RNase cocktail (RNase A, and RNase T1) in either Diluent 54 or Lysis Buffer (MSD Diluent 54+2% LDS+0.4 mg/mL Proteinase K.) was added to the plate and incubated for 30 min at either 30° C. or 37° C., while shaking.

The ECL signals for the SS and AS strands are shown in FIG. 5B. The probes against both strands had a higher sensitivity of detection (lower LLOD) at 30° C. than the probes against both strands at 37° C. (FIG. 5A). The results in FIG. 5B again demonstrate higher sensitivity of detection of both SS and AS strands of the siRNA with the use of lysis buffer at 30° C. hybridization temperature.

### Example 3 siRNA Detection: RNase Treatment at 30° C.

Briefly, individual antisense (AS) or sense (SS) strands of an siRNA were prepared and a 8-point calibration curve was generated for the individual antisense or sense strands by spiking the siRNA into Diluent 54 with a highest calibrator concentration of 800 pM using 4-fold serial dilutions as shown in FIG. 5C.

The detection limits for the sense (SS) and antisense (AS) strands of a model siRNA oligonucleotide (ASO) were determined at two different hybridization temperatures.

A 96-well N-PLEX® plate (Meso Scale Discovery, Rockville, MD, USA (“MSD”)), on which single stranded capture oligonucleotides (with sequences complementary to the oligonucleotide tag sequences of the chimeric sense and antisense probes) were immobilized, was blocked with N-PLEX™ blocking buffer (MSD) and washed 3× with a minimum of 150 μL/well Dulbecco's phosphate-buffered saline (DPBS). After the probes were hybridized to their target strands, the probe/analyte complexes were diluted in buffer, added to the plate and allowed to hybridize for 1 hour at either 30° C. or 37° C., while shaking.

The plates were washed again (3× with a minimum of 150 μL/well DPBS) and an RNase cocktail (RNase A, and RNase T1) in either Diluent 54 or Lysis Buffer (MSD Diluent 54+2% LDS+0.4 mg/mL Proteinase K.) was added to the plate and incubated for 30 min at either 30° C. or 37° C., while shaking.

The ECL signals for the SS and AS strands are shown in FIG. 5C. The probes against both strands had a higher sensitivity of detection (lower LLOD) at 30° C. RNase treatment than the probes against both strands at 37° C. RNase treatment (FIG. 5B). The results in FIG. 5C again demonstrate higher sensitivity of detection of both SS and AS strands of the siRNA with the use of lysis buffer at 30° C.

### Example 4 Extending RNase Incubation Time

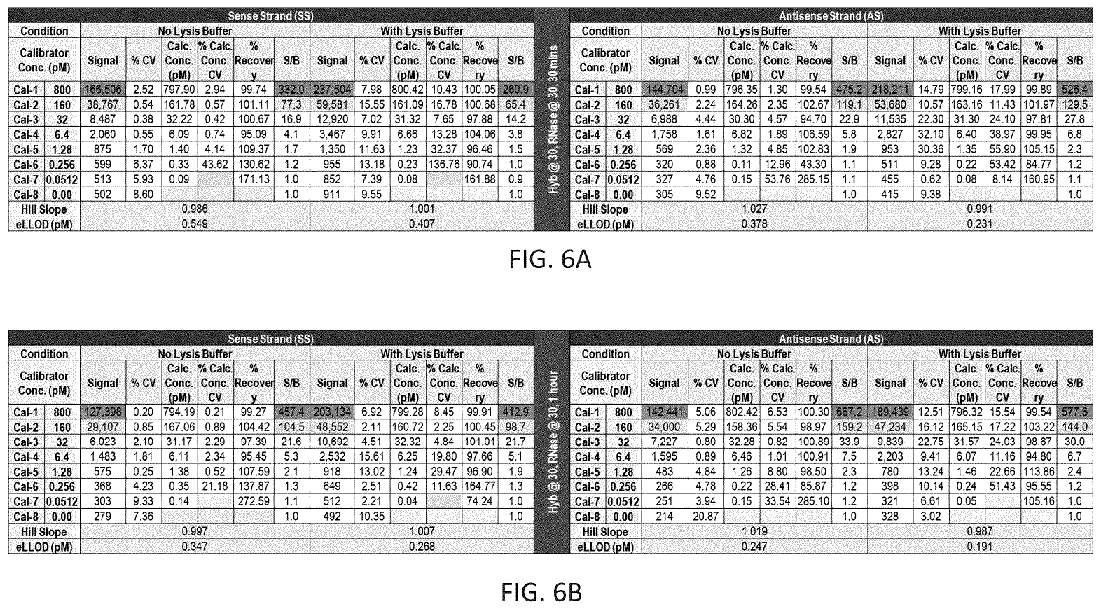

Briefly, individual antisense (AS) or sense (SS) strands of an siRNA were prepared and an 8-point calibration curve was generated for the individual antisense or sense strands by spiking the siRNA into Diluent 54 with a highest calibrator concentration of 800 pM using 4-fold serial dilutions as shown in FIG. 6.

The detection limits for the sense (SS) and antisense (AS) strands of a model siRNA oligonucleotide (ASO) were determined at two different RNase incubation times: 30 min and 1 h.

A 96-well N-PLEX® plate (Meso Scale Discovery, Rockville, MD, USA (“MSD”)), on which single stranded capture oligonucleotides (with sequences complementary to the oligonucleotide tag sequences of the chimeric sense and antisense probes) were immobilized, was blocked with N-PLEX™ blocking buffer (MSD) and washed 3× with a minimum of 150 μL/well Dulbecco's phosphate-buffered saline (DPBS). After the probes were hybridized to their target strands, the probe/analyte complexes were diluted in buffer, added to the plate and allowed to hybridize for 1 hour at 30° C., while shaking.

The plates were washed again (3× with a minimum of 150 μL/well DPBS) and an RNase cocktail (RNase A, and RNase T1) in either Diluent 54 or Lysis Buffer (MSD Diluent 54+2% LDS+0.4 mg/mL Proteinase K.) was added to the plate and incubated for either 30 min or 1 h, at 30° C. while shaking.

The results in FIG. 6A (30 min) and FIG. 6B (1 h) demonstrate extending RNase incubation time from 30 mins to 1 hour further improves detection sensitivity. Extending RNase incubation time along with use of lysis buffer for hybridization and incubation temperature of 30° C. (for both hybridization and RNase treatment) led to higher detection sensitivity.

### Example 5. siRNA Detection Using Shortened Probes

The detection limits for the sense (SS) and antisense (AS) strands of a model 20-mer siRNA were determined. Shortened probes, 16-mer, 17-mer and 18-mer, were used to detect both SS and AS strands of the siRNA analyte either in singleplex detection i.e., SS and AS detected in different wells, or in multiplex detection i.e., SS and AS detected in the same well, using the improved conditions described in Examples 1-4.

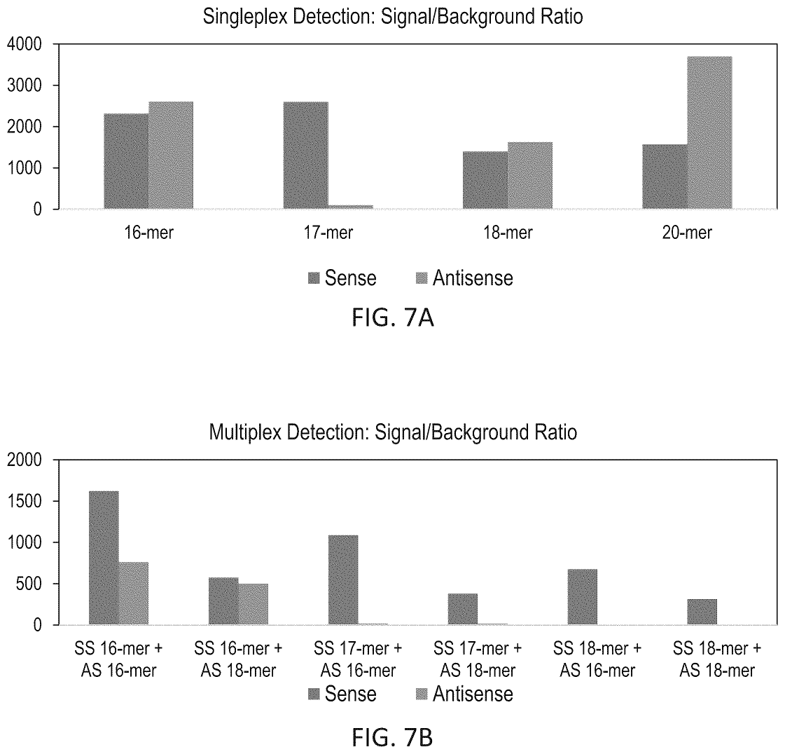

siRNA SS and AS strand detection in the singleplex assay (FIG. 7A and Table 2) show that all shortened probes were capable of sensitive singleplex detection except for AS 17-mer. Note: 20-mer (FL) probes cannot be used in multiplex detection. siRNA SS and AS strand detection in the multiplex assay (FIG. 7B and Table 3) show 16-mer SS+16-mer AS demonstrating ultrasensitive multiplex detection of SS and AS in the multiple probe combinations screened.

TABLE 2
siRNA SS and AS strand singleplex detection

| Strand | Probe | eLLOD (pM) |
|---|---|---|
| SS | 16-mer | 0.166 |
| AS | 16-mer | 0.094 |
| SS | 17-mer | 0.179 |
| AS | 17-mer | 5.254 |
| SS | 18-mer | 0.386 |
| AS | 18-mer | 0.252 |
| SS | 20-mer (FL) | 0.190 |
| AS | 20-mer (FL) | 0.072 |

TABLE 3
siRNA SS and AS strand multiplex detection

| | | eLLOD | Avg eLLOD |
|---|---|---|---|
| Strand | Probe | (pM) | (pM) |
| Sense | SS 16-mer + AS 16-mer | 0.160 | 0.157 |
| Antisense | SS 16-mer + AS 16-mer | 0.154 | |
| Sense | SS 16-mer + AS 18-mer | 0.181 | 0.337 |
| Antisense | SS 16-mer + AS 18-mer | 0.492 | |
| Sense | SS 17-mer + AS 16-mer | 0.151 | 6.036 |
| Antisense | SS 17-mer + AS 16-mer | 11.920 | |
| Sense | SS 17-mer + AS 18-mer | 0.148 | 2.458 |
| Antisense | SS 17-mer + AS 18-mer | 4.767 | |
| Sense | SS 18-mer + AS 16-mer | 0.233 | 16.260 |
| Antisense | SS 18-mer + AS 16-mer | 32.286 | |
| Sense | SS 18-mer + AS 18-mer | 0.221 | 9.361 |
| Antisense | SS 18-mer + AS 18-mer | 18.501 | |

### Example 6. siRNA Multiplex Detection: Assay Reproducibility

The detection limits for the antisense (AS) or sense (SS) strands of the model 20-mer siRNA from Example 5 were determined when spiked in mouse plasma, using the 16-mer AS and SS probes for multiplex detection and the improved conditions described in Examples 1-4.

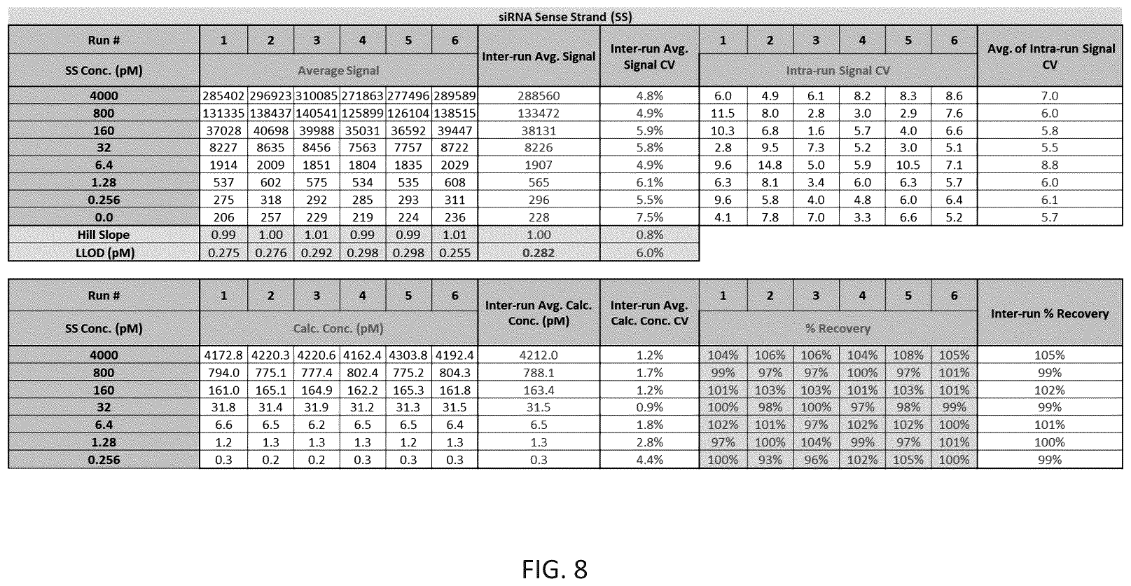

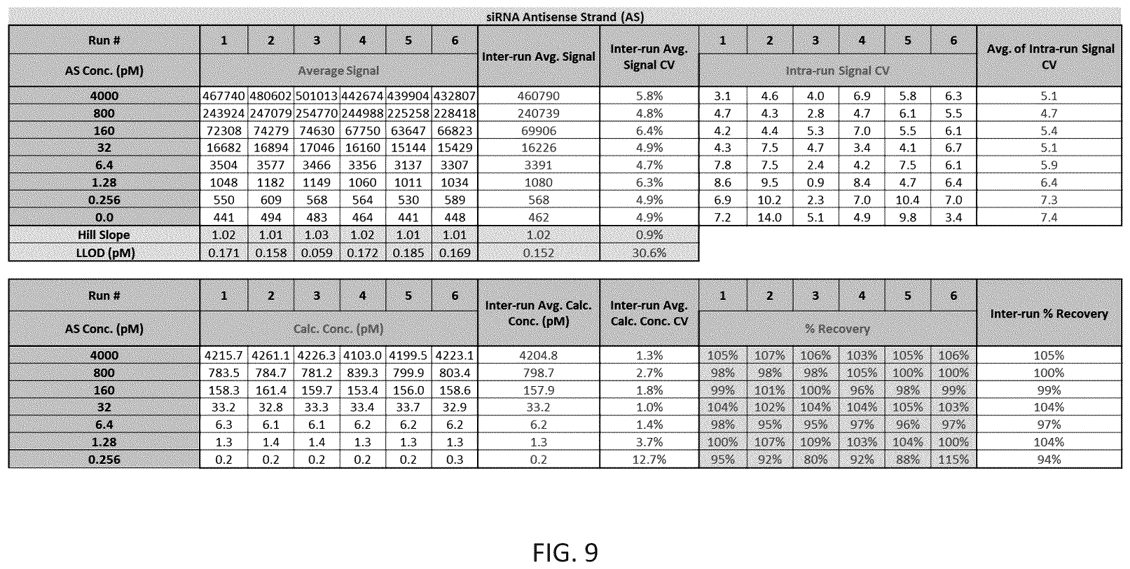

Briefly, a 8-point calibration curve was generated using multiplexed detection of antisense or sense strands of the siRNA with a highest calibrator concentration of 4000 pM using 5-fold serial dilutions, as shown in FIG. 8 (SS) and FIG. 9 (AS). The probes were hybridized to the antisense or sense strands in plasma in duplicate wells.

The results in FIG. 8 and FIG. 9 demonstrate the assay is highly reproducible across 6 independent runs for 3 consecutive days (2 runs/day) for multiplex detection of both SS and AS strands in mouse plasma, with: LLOD for SS=0.282 pM and LLOD for AS=0.152 pM.

### Example 7. siRNA Multiplex Detection: Spike Recovery in Mouse Plasma

Recoveries of high-, mid-, and low-spikes of the antisense (AS) or sense (SS) strands of the model 20-mer siRNA from Example 5 were determined when spiked in mouse plasma, using the 16-mer AS and SS probes for multiplex detection and the improved conditions described in Examples 1-4.

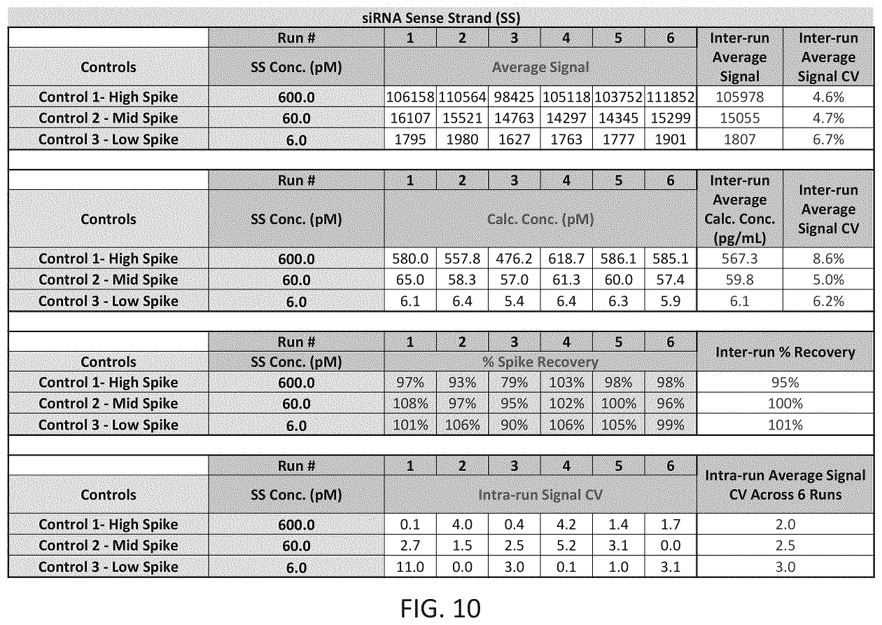

The results in FIG. 10 (SS) demonstrate recoveries of high-, mid- and low-spikes in mouse plasma ranging from 95-101% for detection of siRNA sense strand (SS) and measured across 6 independent runs on 3 consecutive days (2 runs/day).

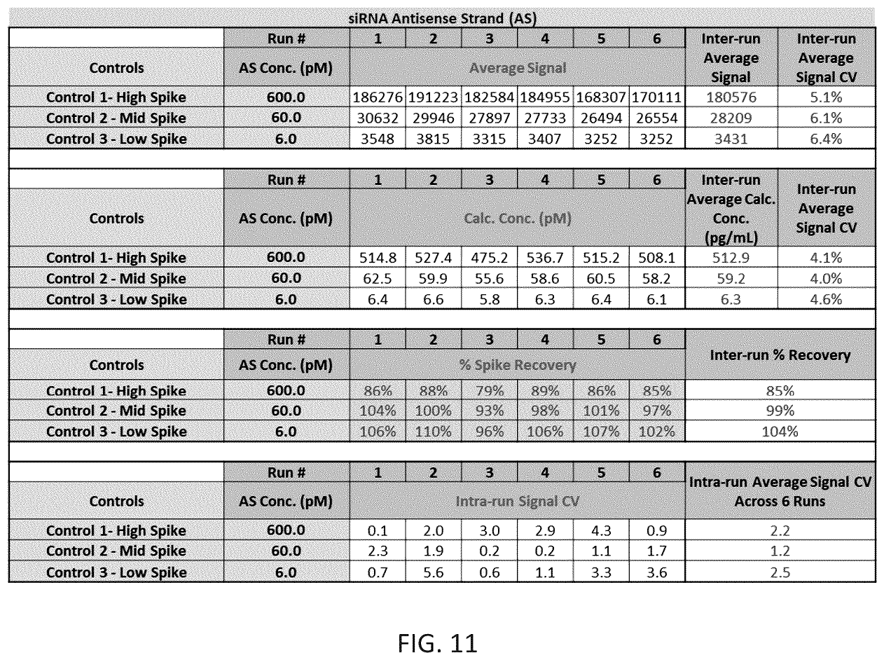

The results in FIG. 11 (AS) demonstrate recoveries of high-, mid- and low-spikes in mouse plasma ranging from 85-104% for detection of siRNA antisense strand (AS) and measured across 6 independent runs on 3 consecutive days (2 runs/day).

### Example 8. siRNA Multiplex Detection: LLOQ Estimation in Mouse Plasma

The detection limits for the antisense (AS) or sense (SS) strands of the model 20-mer siRNA from Example 5 were determined when spiked in mouse plasma, using the 16-mer AS and SS probes for multiplex detection and the improved conditions described in Examples 1-4.

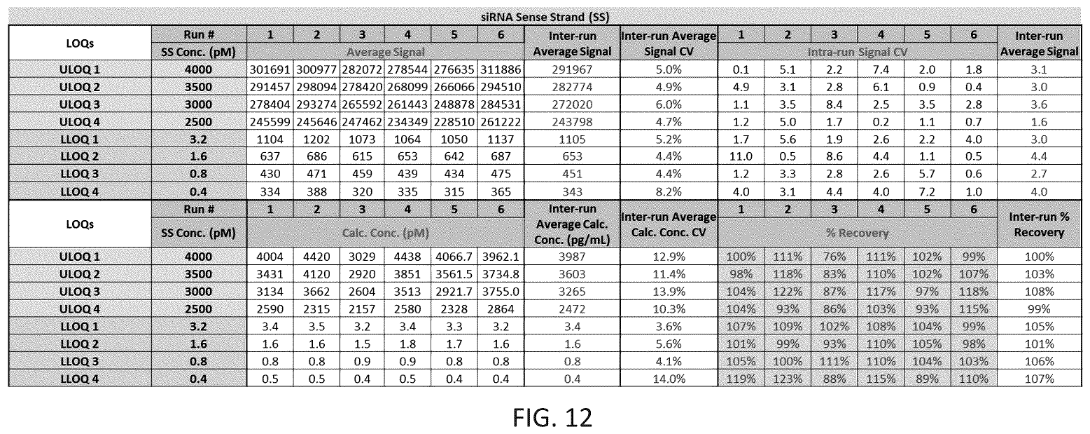

The results in FIG. 12 (SS) show an experimentally determined LLOQ of 0.8 pM (LLOQ3) for siRNA sense strand (SS), measured across 6 independent runs on 3 consecutive days (2 runs/day) in mouse plasma.

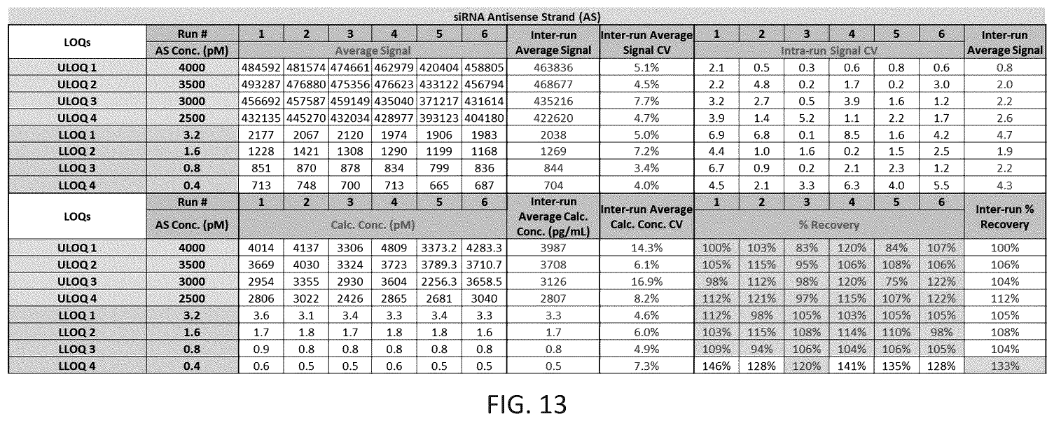

The results in FIG. 13 (AS) show an experimentally determined LLOQ of 0.8 pM (LLOQ3) for siRNA antisense strand (AS), measured across 6 independent runs on 3 consecutive days (2 runs/day) in mouse plasma.

### Example 9. siRNA Multiplex Detection: Linearity of Dilution

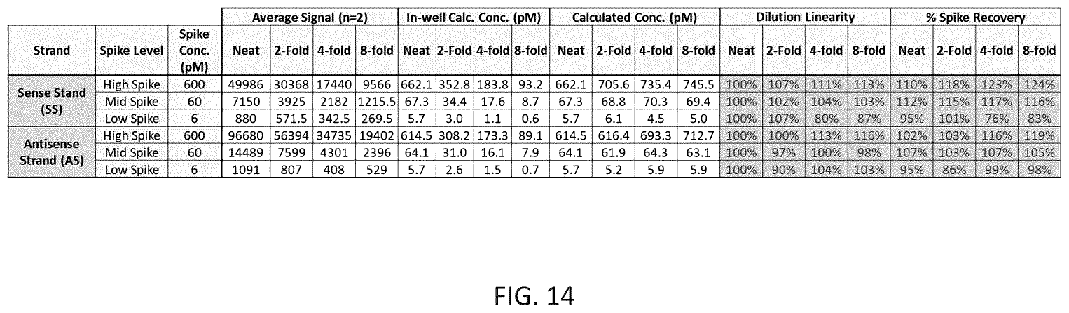

Recoveries of high-, mid-, and low-spikes of the antisense (AS) or sense (SS) strands of the model 20-mer siRNA from Example 5 were determined when spiked in mouse plasma, using the 16-mer AS and SS probes for multiplex detection and the improved conditions described in Examples 1-4. Dilution linearity was computed as shown in FIG. 14, at 2-fold, 4-fold, and 8-fold dilutions for both SS and AS strands. The results demonstrate that the assay shows exemplary linearity of dilution for both SS and AS strands, in mouse plasma.

### Example 10. siRNA Multiplex Detection: Matrix Testing in Mouse Liver and Brain

Recoveries of high-, mid-, and low-spikes of the antisense (AS) or sense (SS) strands of the model 20-mer siRNA from Example 5 were determined when spiked in mouse liver and brain tissue lysates, using the shortened 16-mer AS and SS probes for multiplex detection and the improved conditions described in Examples 1-4. Spike recoveries were measured at three different matrix dilutions: 1:100, 1:1000 and 1:10000.

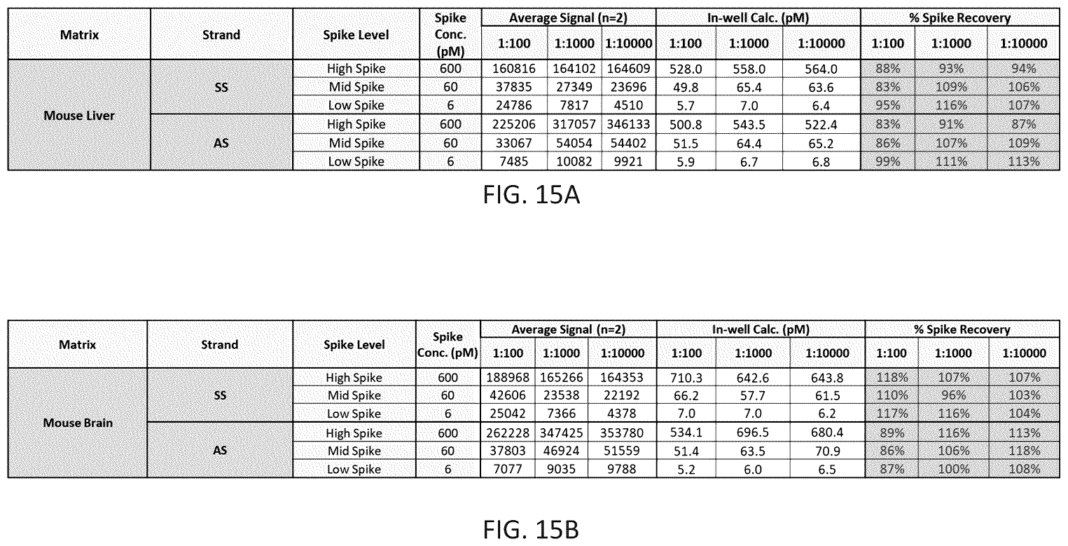

Results in FIG. 15A (liver) and FIG. 15B (brain) demonstrate high recoveries of high-, mid- and low-spikes in mouse liver and brain tissue lysates ranging between 83-118% for both SS and AS strands.

1. A method of detecting or quantifying a sense and an antisense strand of an oligonucleotide duplex in a sample, the method comprising:
(a) contacting the sample with a composition comprising a set of probes to form a hybridization mixture comprising a first hybridization complex between the sense strand and a sense probe and a second hybridization complex between the antisense strand and antisense probe, wherein the set of probes comprises:
(i) a sense probe comprising a first single stranded oligonucleotide tag that is complementary to at least a portion of a first capture oligonucleotide immobilized on a support surface, a sense binding portion capable of hybridizing to a nucleotide sequence of the sense strand of the oligonucleotide duplex, and a first label; and
(ii) an antisense probe comprising a second single stranded oligonucleotide tag that is complementary to at least a portion of a second capture oligonucleotide immobilized on the support surface, an antisense binding portion capable of hybridizing to a nucleotide sequence of the antisense strand of the oligonucleotide duplex, and a second label,
wherein the sense binding portion of the sense probe is at least two nucleotides shorter than a sense strand length of the sense strand, and
wherein the antisense binding portion of the antisense strand is at least two nucleotides shorter than an antisense strand length of the antisense strand; and
(b) contacting the support surface with the hybridization mixture in which the first and second oligonucleotide tags of the sense and antisense probes hybridize to the first and second capture oligonucleotides immobilized on the support surface and contacting the first and second hybridization complex in the hybridization mixture with a single-strand specific nuclease;
wherein the single-strand specific nuclease comprises an RNase; and
(c) detecting or quantifying the sense and antisense strands of the oligonucleotide duplex based on the presence of the label on the support surface.

2. The method of claim 1, wherein step (b) is performed in the presence of a lysis buffer.

3. The method of claim 1 or 2, wherein the oligonucleotide duplex is an RNA duplex.

4. The method of claim 3, wherein the RNA duplex comprises about 17 to about 29 nucleotides.

5. The method of any of claims 1-4, wherein the lower limit of quantitation (LLOQ) is less than about 20 pM for detecting both the sense and antisense strands of the RNA duplex.

6. The method of any of claims 1-5, wherein the lower limit of detection (LLOD) is less than about 1 pM for both the sense and antisense strands of the RNA duplex.
